SEQUENCE LISTING (2 sequences)

SEQ ID NO: 1 - protein, 13 aa, Artificial Sequence
  synthetic peptide
VHFFKNIVTPRTP

SEQ ID NO: 2 - protein, 14 aa, Artificial Sequence
  synthetic peptide
XVHFFKNIVTPRTP

(19) United States
(12) Patent Application Publication
(10) Pub. No.: US 2012/0220537 A1
(43) Pub. Date: Aug. 30, 2012

(54) 9-AMINOACRIDINE DERIVATIVES, THEIR PREPARATION AND USES

(75) Inventor: Gary Gellerman, Rishon Lezion (IL)

(73) Assignee: ARIEL-UNIVERSITY RESEARCH AND DEVELOPMENT COMPANY LTD., Ariel (IL)

(21) Appl. No.: 13/505,436

(22) PCT Filed: Nov. 1, 2010
(86) PCT No.: PCT/IL2010/000905

§ 371 Date: May 1, 2012

Related U.S. Application Data
(60) Provisional application No. 61/256,992, filed on Nov. 1, 2009.
(60) Provisional application No. 61/379,502, filed on Sep. 2, 2010.

(51) Int. Cl. A61K 31/473; C07D 401/12; A61P 35/00; A61K 31/506; A61K 38/10; C07D 219/10; C07K 7/08

(52) U.S. Cl. 514/19.3; 546/105; 546/106; 544/331; 530/327; 514/297; 514/275

(57) ABSTRACT

N-substituted 9-aminoacridine and bis-acridino derivatives containing electron-withdrawing groups (EWG) or electron-donating groups (EDG), including amino acid residues, and one-pot methods for their synthesis are disclosed. The derivatives are potential candidates for cancer treatment.

### FIELD OF THE INVENTION

The present invention relates to new organic compounds, more particularly to derivatives of 9-aminoacridines, their synthesis and uses thereof.

### BACKGROUND OF THE INVENTION

The 9-aminoacridine core is a structure of interest for medicinal chemistry and appears in many biologically active compounds, mostly in anticancer and anti-malaria applications. 9-Aminoacridine derivatives, such as quinacrine, are able to intercalate into DNA and consequently can inhibit DNA transcription in parasites. 9-Anilinoacridines have good antimalarial activities and are potent parasite DNA topoisomerase II inhibitors. N-Alkylated 9-aminoacridine analogs have been shown to be potent inhibitors of prion disease in cultured neuroblastoma cells, which also showed inhibition by lysosomotropic agents and cysteine protease inhibitors.

In the field of antitumor DNA-intercalating agents, 9-aminoacridine derivatives play an important role due to their antiproliferative properties. Several cancer chemotherapeutics based on the 9-aminoacridine core, such as amascrine and ledakrin, have been developed. In addition, potential topoisomerase II-mediated anticancer 9-anilinoacridines designed to avoid bio-oxidation and exhibiting long duration of drug action, have been reported. Among these substances, 3-(9-acridinylamino)-5-hydroxymethyl aniline (AHMA) and its alkylcarbamate derivatives have been developed for clinical applications.

9-Aminoacridine derivatives have also been investigated as potential photoaffinity labels and as fluorescent probes for detection of cancer cells.

Recently, 9-aminoacridine derivatives including the antimalaria drug quinacrine, were found to present a strong induction of p53 function in renal cell carcinoma (RCC) and other types of cancer cells. Interestingly, induction of p53 function by these compounds does not involve genotoxic stress and is mediated by suppression of NF-κB activity. Active NF-κB signaling provides selective advantages to tumor cells by inhibiting apoptosis and promoting proliferation by stimulating expression of antiapoptotic factors.

So far, 9-aminoacridine derivatives have been prepared through several step synthesis involving harsh conditions and laborious purification of intermediates and final compounds. Thus, finding short and efficient methods for the rapid generation of new 9-aminoacridine core-based compounds will greatly enhance their availability for examination in biological systems.

### SUMMARY OF THE INVENTION

In accordance with the present invention, novel 9-aminoacridine derivatives are prepared by simple “one-pot” synthetic approaches.

The present invention relates to a 9-aminoacridine derivative of the formula I or II:

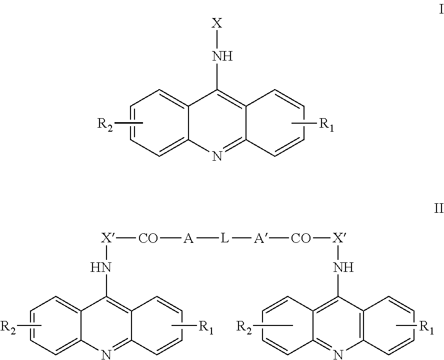

wherein

R₁ and R₂, the same or different, each is H or 1 to 2 substituents selected from electron withdrawing groups (EWG), electron donating groups (EDG), or both;

X is selected from:

(i) —CH₂-aryl or —CH₂-heteroaryl, wherein the aryl or heteroaryl is unsubstituted or substituted with one or more identical or different EWG, EDG, or both;

(ii) aryl substituted with at least one EWG and optionally further substituted with one EDG;

(iii) heteroaryl, unsubstituted or substituted with one or more EWG, one EDG, or both; or

(iv) benzoquinone or a polycyclic aromatic quinone, unsubstituted or substituted with one or more EWG, EDG, or both;

X′ is aryl or heteroaryl substituted with at least one EWG, or —CH₂-aryl or —CH₂-heteroaryl unsubstituted or substituted with one or more identical or different EWG, EDG, or both;

A and A′, the same or different, each is —NH— or —O—; and

L is a linear or branched (C₁-C₁₀)alkylene chain that may be non-adjacently interrupted by one or more N atoms or by a phenylene group, and is optionally substituted by —(CH₂), —Y, wherein n is from 0 to 10 and Y is —OH, —SH, —NH₂, —COOH, CONH₂, an amino acid residue, a (poly)peptide residue, or a polyamine residue —NH—B—NH₂, wherein B is a C₁-C₁₀ alkylene chain optionally non-adjacently interrupted by one or more N atoms;

the EWG may be selected from the groups F, Br, C₁, NO₂, CN, CF₃, SO₃H and COR₃, wherein R₃ is selected from OH; CH₂OH; (C₁-C₁₀)alkoxy; aryloxy; heteroaryloxy; a PEG moiety which may be a PEG moiety of molecular weight in the range of 200 to 40,000 Da, preferably 8,000, 10,000, or 20,000 Da; (C₁-C₁₀) alkyl; aryl; heteroaryl; a residue of an amino acid or of a derivative thereof, linked to the CO group through its α-amino group; NH₂; or a polyamine residue of the formula —NH—B—NH₂, wherein B is a C₁-C₁₀ alkylene chain optionally non-adjacently interrupted by one or more N atoms;

the EDG may be selected from (C₁-C₁₀)alkyl, OH, (C₁-C₁₀)alkoxy, or —N(R₄R₅), wherein R₄ and R₅ each independently is H or (C₁-C₁₀)alkyl;

and pharmaceutically acceptable salts thereof.

The present invention further provides novel synthetic methods for the preparation of the 9-aminoacridine derivatives of the formula I or II above as described herein in the specification.

In addition, the present invention relates to a pharmaceutical composition comprising a 9-aminoacridine derivative of the formula I or II or a pharmaceutically acceptable salt thereof, and a pharmaceutically acceptable carrier.

### BRIEF DESCRIPTION OF THE FIGURES

Some embodiments of the invention are described herein with reference to the accompanying figure and schemes. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention.

FIGS. 1A-1B show the antiproliferative effect of the antineoplastic drug amsacrine and of the compound of Formula I 2-(acridin-9-ylamino)-3-ethoxy-5,8-dihydroxynaphtoquinone (Compound 41 herein), respectively, on various cancer cell lines (solid black bars represent 0 μg/ml of amsacrine or of Compound 41; solid white bars represent 0.5 μg/ml of amsacrine or of Compound 41; solid gray bars represent 5 μg/ml of amsacrine or of Compound 41; and hatched bars represent 50 μg/ml of amsacrine or of Compound 41).

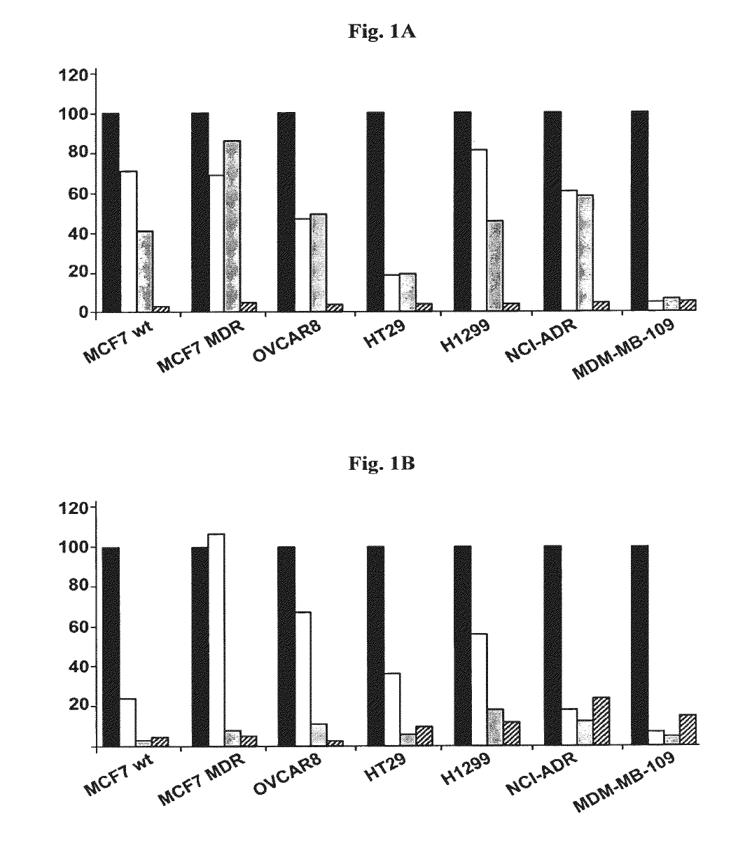

### DETAILED DESCRIPTION OF THE INVENTION

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Unless otherwise defined, all technical and scientific terms used herein have the same meaning as commonly understood by one of ordinary skill in the art to which the invention pertains. In case of conflict, the specification, including definitions, will control.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

In the description and Examples herein, the 9-aminoacridine derivatives of the invention will be represented in bold by their respective Arabic numbers (1-51) preceded by the word Compound. The full structural formulas of Compounds 1-51 are presented in Appendix I at the end of the description, just before the Schemes. The Schemes 1-10 depicting the methods for the preparation of compounds of the invention are present before Claims.

As used herein, the term “C₁-C₁₀ alkyl”, alone or as part of a radical containing an alkyl group, typically means a straight or branched radical having 1 to 10, preferably 1 to 6, more preferably 5, 4, 3, 2 or 1 carbon atoms and includes, without being limited to, methyl, ethyl, n-propyl, isopropyl, n-butyl, sec-butyl, isobutyl, tert-butyl, n-pentyl, 1-methylbutyl, 2,2-dimethylpropyl, n-hexyl, n-heptyl, n-octyl, n-nonyl and n-decyl, and the like. The term “C₁-C₁₀ alkylene” typically means a straight or branched radical derived from an alkane having 1 to 10 carbon atoms, preferably 1 to 6, more preferably 5, 4, 3, 2 or 1 carbon atoms, which is substituted at both ends, and includes, without being limited to, methylene, ethylene, n-propylene, isopropylene, n-butylene, sec-butylene, isobutylene, n-pentylene, 1-methylbutylene, 2,2-dimethylpropylene, n-hexylene, n-heptylene, n-octylene, n-nonylene and n-decylene, and the like.

The term “C₁-C₁₀ alkoxy” as used herein typically means a straight or branched radical having 1-10, preferably 1, 2, or 3 carbon atoms, such as methoxy, ethoxy, propoxy, isopropoxy, butoxy, pentoxy, and the like. In some preferred embodiments the alkoxy is methoxy or ethoxy.

The term “aryl” as used herein refers to a mono or bicyclic aromatic carbocyclic group such as, but not limited to, phenyl and naphthyl. In some preferred embodiments, the aryl is phenyl.

The term “heteroaryl” as used herein refers to a mono or bicyclic heteroaromatic group, in which at least one of the rings is a 5- or 6-membered ring containing 1-3 N atoms which may be condensed to a benzo ring or to another 6-membered ring containing 1-3 N atoms. Examples of heteroaryl according to the invention include, but are not limited to, pyrrolyl, imidazolyl, pyridyl, pyrimidyl, triazinyl such as 1,3,5-triazinyl, indolyl, quinolyl, isoquinolyl, benzopyrimidyl (quinazolyl), benzopyrazyl (quinoxalyl), and pyridopyridyl (naphthyridyl). In some preferred embodiments, the heteroaryl is pyridyl or pyrimidyl.

The term “electron withdrawing group” or “EWG” as used herein refers to a group that draws electrons away from a reaction center such as from an aromatic ring. Examples of EWG include halogens (F, Cl, Br), CF₃, cyano (CN), nitro, SO₃H and groups containing CO such as COOH, ester groups such as CO-alkoxy, for example, COOMe or CO-PEG, amide group, i.e., CONH₂, or a ketone group such as —CO-alkyl, CO-phenyl or CO-heteroaryl.

The term “electron donating group” or “EDG” (also called electron releasing groups) as used herein refers to a group that releases electrons into a reaction center. Examples of EDG include OH, NH₂, alkyl as defined hereinabove, for example, methyl, and alkoxy as defined hereinabove, for example, methoxy and ethoxy.

The term “amino acid” as used herein is understood to include the amino acids selected from the 20 naturally occurring α-amino acids as well as natural amino acids that are less abundant or non-natural amino acids. In some embodiments, the amino acid is glycine. The term “amino acid” includes both D- and L-amino acids. In some preferred embodiments, the amino acid is a trifunctional amino acid. The term “trifunctional amino acid” as used herein is understood to include the amino acids selected from the 20 naturally occurring α-amino acids as well as natural amino acids that are less abundant or non-natural amino acids, which have an amino, a carboxy and a further functional group such as OH, SH, NH₂, COOH, CONH₂, or guanidino. In some embodiments, the trifunctional amino acid is serine, lysine or arginine.

The term “derivatives thereof” refers to a chemical derivative of the amino acid including, but not limited to, a derivative containing additional chemical moiety not normally a part of the amino acid, in particular a chemical derivatization of the free functional group. Examples of amino acid chemical derivatives include, for example, amides (—CONH₂) or esters (—COOR, wherein R is alkyl, aryl or heteroaryl as defined herein) of the C-terminus COOH or of an additional COOH group of a trifunctional amino acid, and N-acylated (N—COR, wherein R is alkyl, aryl or heteroaryl as defined herein) derivatives of the N-terminus —NH₂ or of an additional —NH₂ group of a trifunctional amino acid.

The term “peptide” as used herein refers to a molecule comprising two or more amino acids joined by a peptide bond. The peptides used in the present invention can vary in length from di-peptides with two amino acids to polypeptides with several hundred amino acids. They may be homo- or hetero-peptides and can include natural amino acids, synthetic amino acids, or any combination thereof. In some embodiments, the peptide is the myelin basic protein peptide amide (MBPP) of the sequence set forth in SEQ ID NO:1 or a derivative thereof with an additional O-Ala residue at the N-terminus of the sequence set forth in SEQ ID NO:2 herein.

In some embodiments, in the 9-aminoacridine derivative of formula I or II of the invention the aryl is phenyl or naphthyl; heteroaryl is a mono or bicyclic group in which at least one of the rings is a 5- or 6-membered ring containing 1-3 N atoms which may be condensed to a benzo ring or to another 6-membered ring containing 1-3 N atoms such as pyrrolyl, imidazolyl, pyridyl, pyrimidyl, triazinyl, indolyl, quinolyl, isoquinolyl, benzopyrimidyl, benzopyrazyl, and pyridopyridyl, preferably pyridyl or pyrimidyl; the quinone is benzoquinone or naphthoquinone; and the amino acid is selected from the 20 naturally occurring α-amino acids, natural amino acids that are less abundant or non-natural amino acids, or a chemical derivative thereof that may be an ester or amide of the carboxy group or an N-acyl derivative of the amino group.

In some embodiments, the 9-aminoacridine derivative of the invention has the formula I, wherein R₁ and R₂ are both H, and X is selected from:

(i) —CH₂-phenyl, wherein the phenyl is substituted with one EWG that may be COOH or nitro, or one EDG that may be OH, or with or one EWG that may be Br and two identical EDGs that may be methoxy, or with three identical EDGs that may be methyl or methoxy groups;

(ii) —CH₂-naphthyl substituted with one EDG that may be OH;

(iii) —CH₂-indolyl substituted with one EDG that may be methoxy;

(iv) phenyl substituted with one EWG that may be a nitro group; with two identical EWGs that may be nitro groups; with two different EWGs wherein one EWG is a nitro group and the other EWG is COOH, CONH₂, CN, or SO₃H, or one EWG is CF₃ and the other EWG is COOMe; or with one EWG that may be a nitro group and one EDG that may be OH or methoxy;

(v) pyridyl, unsubstituted or substituted with one EWG that may be a nitro or CN group;

(vi) pyrimidyl, unsubstituted or substituted with one EWG that may be Br;

(vii) benzoquinone substituted with one EWG that may be Br and one EDG that may be ethoxy; with two EWGs that may be Cl and one EDG that may be ethoxy; or with three EWGs that may be Br;

(vii) naphthoquinone substituted with one EWG that may be Cl; with one EWG that may be Cl and two EDGs that may be methyl; or with three EDGs, wherein one of them may be ethoxy and the other two EDGs are identical and may be OH; or

(viii) phenyl substituted with two different EWGs, wherein one EWG may be nitro and the other EWG may be a group COR₃, wherein R₃ is a residue of an amino acid or of a derivative thereof.

In some embodiments, the 9-aminoacridine derivative of the invention is a compound of formula I in which R₁ and R₂ are H and X is —CH₂-aryl or —CH₂-heteroaryl, wherein said aryl or heteroaryl is unsubstituted or substituted with one or more identical or different EWG, EDG, or both. In some embodiments, X is —CH₂-phenyl substituted solely with one EWG, for example, COOH at position 2 (Compound 1) or 4 (Compound 2 herein) of the phenyl ring. In some other embodiments, the phenyl ring is substituted with one EWG, for example, NO₂ or COOH, at position meta and one EDG, for example, methyl, hydroxyl, methoxy, or halogen, preferably at position ortho to the —CH₂— group. In a particular embodiment, the one EWG is NO₂ at position meta and the one EDG is OH at position ortho (Compound 3 herein) and, in another embodiment, the NO₂ is at position ortho and the OH is at position meta (Compound 4 herein). In some other embodiments, the phenyl ring is substituted solely with one or more identical or different EDGs, for example, alkyl, alkoxy, or halogen, for example, 3 EDG groups such as three methyl groups at positions 2,4,6 (Compound 5 herein), three methoxy groups at positions 3,4,5 (Compound 6 herein), or two methoxy groups at positions 4,5 and one Br atom at position 2 (Compound 7 herein).

In some embodiments, the 9-aminoacridine derivative of the invention is a compound of formula I in which R₁ and R₂ are H and X is —CH₂-naphthyl substituted solely with one EDG, for example, hydroxyl, preferably at position ortho to the —CH₂— group (Compound 8).

In some other embodiments, the 9-aminoacridine derivative of the invention is a compound of formula I in which R₁ and R₂ are H and X is —CH₂-heteroaryl, wherein the heteroaryl is as defined herein above and is, for example, 3-indolyl substituted at position 5 with an EDG group, preferably methoxy (Compound 9 herein).

In some embodiments of the invention, the 9-aminoacridine derivative is a compound of formula I in which R₁ and R₂ each is H and X is aryl substituted with at least one EWG and optionally further substituted with one EDG. In some embodiments, X is phenyl substituted solely with one EWG, for example, NO₂ at position ortho or para to the —NH— group (Compounds 10 and 11 herein, respectively). In some other embodiments, the phenyl ring is substituted with two EWGs, for example, two NO₂ groups that may be at positions ortho and para to the —NH— group (Compound 12 herein), or one NO₂ that may be at position ortho and one COOH or CONH₂ that may be at position para to the —NH— group (Compound 13 and 14 herein, respectively), or one CF₃ and one COOCH₃ that may be at positions ortho and para, respectively, to the —NH— group (Compound 15 herein), or one NO₂ and one CN that may be at positions ortho and para, respectively, to the —NH— group (Compound 16 herein), or one NO₂ and one SO₃H that may be at positions ortho and para, respectively, to the —NH— group (Compound 17 herein). In some other embodiments, X is phenyl substituted with three EWGs, for example, two NO₂ at positions 2 and 4 and one halogen, e.g., F, at position 5 of the phenyl ring (Compound 18 herein). In some other embodiments, X is phenyl substituted with one EWG and one EDG, for example NO₂ at position para and OH at position meta or OCH₃ at position ortho to the —NH— group (Compounds 19 and 20, respectively).

In some embodiments of the invention, the 9-aminoacridine derivative is a compound of formula I in which R₁ and R₂ each is H and X is heteroaryl as defined herein that may be substituted with one or more EWG, a sole EDG, or both. In some embodiments, X is unsubstituted 2-pyridyl (Compound 22), 2-pyridyl substituted with one EWG, for example, NO₂ at position 3 (Compound 21) or CN at position 5 (Compound 23), or 2-pyridyl substituted with two EWG or one EWG and one EDG, for example, NO₂ at position 3 and Cl, CN or CO₂H at position 5 (Compounds 25, 26 and 27, respectively), or NO₂ at position 3 and CH₃ at position 5 (Compound 24). In some other embodiments, X is unsubstituted 2-pyrimidyl (Compound 28), 2-pyrimidyl substituted with one EWG, for example, Br at position 5 (Compound 29), Cl at position 5 (Compound 31), CN at position 5 (Compound 32) or CO₂H at position 5 (Compound 33), or 2-pyrimidyl substituted with one EDG, for example, CH₃ at position 5 (Compound 30).

According to some further embodiments of the invention, the 9-aminoacridine derivative is a compound of formula I in which R₁ and R₂ each is H and X is benzoquinone, preferably 1,4-benzoquinone, that may be substituted with one or more EWG, EDG, or both. In some embodiments, X is 1,4-benzoquinon-2-yl substituted with 1 to 3 EWG, EDG or both. For example, the 1,4-benzoquinon-2-yl may be substituted with two groups such as one EWG e.g. halogen such as Br at position 3 and one EDG such as alkoxy, e.g., ethoxy at position 5 (Compound 35 herein). The 1,4-benzoquinon-2-yl may be also substituted with three groups such as two EWG e.g. halogen such as Cl at positions 3 and 6 and one EDG such as alkoxy, e.g., ethoxy at position 5 (Compound 34 herein), or with three EWGs such as halogen, e.g. Br at positions 3, 5 and 6 (Compound 38 herein).

According to some other embodiments of the invention, the 9-aminoacridine derivative is a compound of formula I in which R₁ and R₂ each is H and X is a polycyclic aromatic quinone that may be substituted in the quinone and also in the one or more aromatic rings with one or more EWG, EDG, or both. In some embodiments, X is preferably 1,4-naphthoquinon-2-yl that may be substituted at position 3 with an EWG, e.g., halogen such as Cl (Compound 39), or with one EWG such as Cl at position 3 and two methyl groups at positions 6,7 (Compound 40 herein) or with three EDGs such as OH and alkoxy. In one more preferred embodiment, the 1,4-naphthoquinon-2-yl is substituted with ethoxy at position 3 and two hydroxyl groups at positions 5 and 8 (Compound 41 herein).

According to some other embodiments of the invention, the 9-aminoacridine derivative is a compound of formula I in which R₁ and R₂ each is H and X is an aryl radical substituted with at least one EWG and optionally with one EDG. In some embodiments, X is phenyl substituted with two EWGs, one EWG is NO₂, for example at position 2, and the other EWG is COR₃, for example at position 4, wherein R₃ is a residue of an amino acid or of a derivative thereof or of a peptide residue.

As mentioned above, the amino acid may be one of the 20 naturally occurring α-amino acids, another natural amino acid that occurs naturally but is less abundant in nature or is a non-natural amino acid. In some embodiments, the amino acid contains no further functional groups such as glycine, alanine, β-alanine, phenylalanine, homophenylalanine, valine, homovaline, leucine, homoleucine, and isoleucine. In some other embodiments, the amino acid is a trifunctional amino acid containing an additional functional group such as OH, SH, NH₂, COOH, CONH₂, or guanidine.

In some embodiments, the trifunctional amino acid has an additional amino group and may be, for example, lysine, ornithine, homolysine, 2,4-diaminobutyric acid (DABA), 2,3-diaminopropionic acid (DAP). In a preferred embodiment, the diamino acid is lysine (Lys). In some embodiments, the trifunctional amino acid has a hydroxyl group and may be serine, homoserine, threonine, or tyrosine. In a preferred embodiment, the hydroxylamino acid is serine (Ser). In some other embodiments, the trifunctional amino acid has an additional carboxy group and may, for example, be aspartic acid (Asp), β-aspartic acid, homoaspartic acid, glutamic acid (Glu), β-glutamic acid, homoglutamic acid. In some embodiments, the trifunctional amino acid has a guanidine group and is, for example, arginine (Arg) or homoarginine. In some other embodiments, the trifunctional amino acid has an additional carboxamide group and may, for example, be asparagine, homoasparagine, β-homoasparagine, glutamine, homoglutamine, or β-homoglutamine. In some other embodiments, the trifunctional amino acid has a SH group and may, for example, be cysteine or homocysteine.

According to some embodiments, R₃ may also be the residue of a derivative of an amino acid. Examples of such derivatives are: (a) N-acyl derivatives of the free amino group of a trifunctional amino acid, wherein the acyl group may be either an alkanoyl group such as acetyl, hexanoyl, octanoyl; an aroyl group, e.g., benzoyl, or biotinyl; (b) esters of the carboxyl terminal or of another free carboxyl of a trifunctional amino acid, for example, C₁-C₁₀ alkyl, phenyl, or benzyl esters, or of hydroxyl groups, for example, O-acyl esters, wherein acyl is as defined hereinabove; and (c) amides of the carboxyl terminal or of another free carboxyl groups, wherein the amino group may be substituted by one or two identical or different C₁-C₁₀ alkyl, phenyl or benzyl groups.

The amino acid or peptide to be added to the 9-aminoacridine molecule may be selected according to the purpose of the invention. For improving solubility, amino acids such as lysine, arginine, aspartic acid or glutamic acid can be chosen; for improving delivery of the drug molecule, amino acids such as glutamine, asparagine, lysine and arginine or the homopeptide polyarginine may be preferred.

Thus, in some embodiments, the 9-aminoacridine derivative of the invention has the

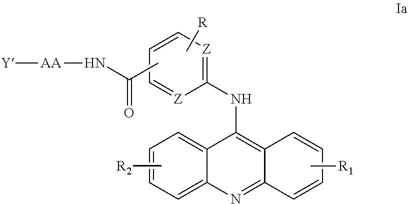

wherein
Z each is CH or N;
R₁ and R₂, the same or different, each is one or two EWG, EDG or both;

R is one or two EWG groups;

AA is the residue of an amino acid or of a derivative thereof, bound to the —CO-linked to the ring via its α-amino group shown as —NH—, and may be a residue of a trifunctional amino acid; and

Y′ is H or, when AA is the residue of a trifunctional amino acid, Y′ is the additional functional group of the trifunctional amino acid and may be —COOH, NH₂, NH—C(═NH)NH₂, OH, SH, CONH₂, or a polyamine group —CO—NH—B—NH₂, wherein B is a C₁-C₁₀ alkylene chain optionally non-adjacently interrupted by one or more N atoms, or a peptide residue.

In the formula Ia above, when each Z is CH, R₁ and R₂ are both H, AA is the residue of L-serine, Y′ is OH, the Y′-AA group is represented by the formula —CH(CH₂OH)—COOH or CH(CH₂OH)—CONH₂ and the compounds are herein identified as Compounds 42 and Compound 43, respectively; when AA is the residue of glycine, Y′ is OH, the Y′-AA group is represented by the formula —NH—CH₂—CONH₂ (Compound 44); when AA is the residue of L-arginine, Y′ is NH₂, the Y′-AA group is represented by the formula —CH—(CONH₂)—CH₂)₃—NH—C(═NH)—NH₂ group and is identified herein as Compound 45; when AA is the residue of L-lysine, Y′ is NH₂, the Y′-AA group is represented by the formula —CH(CONH₂)—(CH₂)₄—NH₂ and is identified herein as Compound 46.

Other derivatives of formula Ia according to the invention wherein AA is the residue of a trifunctional natural, non-abundant or non-natural amino acid are as follows:

(i) with homolysine, AA residue: —CH(CONH₂)—(CH₂)₅—NH₂;

(ii) with ornithine, AA residue: —CH(CONH₂)—(CH₂)₃—NH₂;

(iii) with 2,4-diaminobutyric acid (daba), AA residue: —CH(CONH₂)—(CH₂)₂—NH₂;

(iv) with 2,3-diaminopropanoic acid (dap), AA residue: —CH(CONH₂)—CH₂—NH₂;

(v) with homoserine, AA residue: —NH—CH(CONH₂)—CH₂—CH₂ OH;

(vi) with threonine, AA residue: —NH—CH(CONH₂)—CH(CH₃)—OH;

(vii) with tyrosine, AA residue: —NH—CH(CONH₂)—CH₂-phenyl-OH;

(viii) with homotyrosine, AA residue: —NH—CH(CONH₂)—(CH₂)₂-phenyl-OH;

(ix) with β-homotyrosine, AA residue: —NH—CH(CH₂—CONH₂)-phenyl-OH;

(x) with cysteine, AA residue: —NH—CH(COOH)—CH₂ SH

(xi) with homocysteine, AA residue: —NH—CH(CONH₂)—CH₂—CH₂ SH;

(xii) with aspartic acid, AA residue: —NH—CH(COOH)—CH₂—COOH;

(xiii) with asparagine, AA residue: —NH—CH(COOH)—CH₂—CONH₂;

(xiv) with glutamic acid, AA residue: —NH—CH(COOH)—(CH₂)₂—COOH; and

(xv) with glutamine, AA residue: —NH—CH(COOH)—(CH₂)₂—CONH₂.

The carboxy of the amino acid AA appears as —COOH or —CONH₂. In some embodiments, it may have a polyamine substitution. As used herein, the term “polyamine” refers to an organic compound having two or more primary amino groups —NH₂. This is comprised within the definition —NH—B—NH₂, wherein B is a C₁-C₁₀ alkylene chain optionally non-adjacently interrupted by one or more N atoms, or a peptide residue. For example, the polyamine may be a diamine, in which case B is an alkylene chain, for example, di-, tri-, tetra or hexa-methylene; a triamine, for example, diethylene triamine (—NH—(CH₂)₂—NH—(CH₂)₂—NH₂) or dipropylene triamine (—NH—(CH₂)₃—NH—(CH₂)₃—NH₂); a tetraamine, for example, triethylene tetraamine (—NH—(CH₂)₂—NH—(CH₂)₂—NH—(CH₂)₂—NH₂) or tripropylene tetraamine (—NH—(CH₂)₃—NH—(CH₂)₃—NH—(CH₂)₃—NH₂), and the like.

In some embodiments, the carboxy of the amino acid AA is substituted with a peptide residue, that may contain from 5 to 20 amino acid residues that may be natural or non-natural amino acids as defined hereinabove. In some embodiments, the peptide is a targeting peptide to cancer cells. These 9-aminoacridine-targeting peptide conjugates will direct the 9-aminoacridine-based anticancer drug to the cancer cells to which the peptide targets and binds. Examples of cancer cells targeting peptide include the myelin basic protein peptide (MBPp) of the sequence Val-His-Phe-Phe-Lys-Asn-Ile-Val-Thr-Pro-Arg-Thr-Pro-NH₂ (SEQ ID NO: 1), which specifically targets to the human B-cell malignancy multiple myeloma cells (Cohen S. et al, J, Biol. Chem., 282, pp. 28301-08, 2009) and its analog of SEQ ID NO:2 containing a residue of the naturally less abundant amino acid 3-alanine.

In some other embodiments, the present invention relates to a bis-9-aminoacridine compound of formula II herein in which:

X′ is aryl or heteroaryl substituted with at least one EWG, or —CH₂-aryl or —CH₂-heteroaryl unsubstituted or substituted with one or more identical or different EWG, EDG, or both;

A and A′, the same or different, each is —NH—, —O—, or —S—;

L is a linear or branched (C₁-C₁₀)alkylene chain that may be interrupted by one or more N atoms or by a phenylene group and is optionally substituted with —(CH₂)ₙ—Y, wherein n is from 0 to 10 and Y is —OH, —SH, —NH₂, —COOH, —CONH₂, —CO—NH—B—NH₂, wherein B is a C₁-C₁₀ alkylene chain optionally non-adjacently interrupted by one or more N atoms, or a peptide residue; and

EWG and EDW groups are as defined before for compounds of formula I.

As used herein, the term “(C₁-C₁₀)alkylene chain that may be interrupted by a phenylene group” for the substituent L includes also an alkylene chain terminated by a phenylene chain as for the derivatives with tyrosine, homotyrosine and O-homotyrosine shown herein below.

In some embodiments, the invention relates to a compound of formula II in which R₁ and R₂ each is H, X′ each is an aryl group substituted at the ortho position to the —NH —with an EWG, A and A′ each is —NH—, and L is (C₁-C₁₀)alkylene substituted with —(CH₂)ₙ—Y, wherein n is from 0 to 10 and Y is —OH, —SH, —NH₂, —COOH, —CONH₂, or a peptide residue. In some preferred embodiments, X′ each is phenyl substituted at the ortho position to the —NH— with NO₂, A and A′ each is —NH— and L, at the meta position to the NO₂ group, is a C₄-linear alkylene substituted at the C adjacent to the —NH— with —COOH (identified herein as Compound 47, and obtained using lysine) or with —CONH₂ (identified herein as Compound 48), or L at the meta position to the NO₂ group is a C₂ or C₃-alkylene substituted at the C adjacent to the —NH— with —CONH₂ (Compounds 49 and 50, respectively).

The compounds herein identified as 47 and 48 contain the residue of lysine in which the carboxy group appears as a free group or as amide, respectively. The Compound 49 contain the residue of 2,4-diaminobutyric acid (daba) in which the carboxy group appears as amide, and Compound 50 contain the residue of 2,5-diaminovaleric acid (ornithine) in which the carboxy group appears as amide.

With other trifunctional natural, non-abundant or non-natural amino acid derivatives other compounds according to the invention are obtained with the A-L-A′ group as follows:
(i) with homolysine: —NH—CH(CONH₂)—(CH₂)₅—NH—;
(ii) with 2,3-diaminopropanoic acid (dap): —NH—CH(CONH₂)—CH₂—NH—;
(iii) with homoserine: —NH—CH(CONH₂)—CH₂—CH₂—O—;
(iv) with threonine: —NH—CH(CONH₂)—CH(CH₃)—O—;
(v) with tyrosine: —NH—CH(CONH₂)—CH₂-phenyl-O—;
(vi) with homotyrosine: —NH—CH(CONH₂)—(CH₂)₂-phenyl-O—;
(vii) with β-homotyrosine: —NH—CH(CH₂—CONH₂)-phenyl-O—;
(viii) with cysteine: —NH—CH(COOH)—CH₂—S—;
(ix) with homocysteine: —NH—CH(CONH₂)—CH₂—CH₂—S—;
(x) with aspartic acid: —NH—CH(COOH)—CH₂—CO—O—;
(xi) with glutamic acid: —NH—CH(COOH)—(CH₂)₂—CO—O—.

In some embodiments, Y is a peptide residue, more particularly a targeting peptide to cancer cells as described above. An example of such a conjugate is the compound herein identified as Compound 51, in which each X′ is phenyl substituted at the ortho position to the —NH— with an NO₂ group, A and A′ each is —NH—, and L is C₄ alkylene substituted with —(CH₂)—Y, wherein n is from 0 to 10 and Y is —OH, —SH, —NH₂, —COOH, —CONH₂ or a peptide residue. In some preferred embodiments, X′ each is phenyl substituted at the ortho position to the —NH— with NO₂, A and A′ each is —NH— and L, at the meta position to the NO₂ group, is C₄-alkylene substituted at one of the C adjacent to the —NH— with the above-described analog of the MBP peptide of SEQ ID NO:1 containing as linker a β-Ala residue at the amino terminal (SEQ ID NO:2) and amidated at the C terminal.

In some embodiments, the -A-L-A′- group between the two CO groups in Formula II may be a radical derived from a polyamine as defined hereinabove: with a diamine, for example, A and A′ each is —NH— and L is (C₁-C₁₀)alkylene or phenylene, while with a polyamine A and A′ each is —NH— and L is (C₁-C₁₀)alkylene interrupted by one or more —NH— groups, e.g., one for a triamine, two for a tetraamine, and so forth.

In some embodiments, the -A-L-A′- group between the two CO groups in Formula II may be a radical derived from an amino alcohol, in which case one of A or A′ is —NH— and the other is —O— and L will preferably be an alkylene of 2-10 carbon atoms.

In another aspect, the present invention provides four short and efficient methods for rapid derivatization of 9-aminoacridine scaffolds suitable for generation of new compounds for screening and pharmacological evaluation of new 9-aminoacridine-based drugs, particularly for treatment of cancer. These methods are: (i) reductive amination, (ii) nucleophilic aromatic substitution (herein S_{N}Ar), (iii) addition-elimination (AE), and (iv) solid phase synthesis (SPS). These methods are preferably performed as one-pot derivatizations.

In one embodiment, the method consists in direct reductive amination of aldehydes with a 9-aminoacridine compound in the presence of a suitable reducing agent, resulting in direct transformation of the aldehyde functional group into amine. In this way, 9-aminoacridine derivatives of formula I in which X is —CH₂-aryl or —CH₂-heteroaryl can be obtained in good yields. In this method, 9-aminoacridine is reacted with the corresponding (hetero)aromatic aldehyde in the presence of a suitable reducing agent, using commercially available synthons, resulting in direct transformation of the aldehyde functional group into amine. Suitable reducing agents include NaCNBH₃, NaBH(OAc)₃, Py-BH₃, Me₂S—BH₃, NaBH₄ and diborane alone or with additives such as TiCl₄ and the like, generally in a weak acid medium. In some embodiments, mild NaCNBH₃ in weak acidic media is used as a reducing reagent for reductive amination. Scheme 1 hereinafter depicts the reaction of 9-aminoacridine with benzaldehyde that may be unsubstituted or substituted with one or more identical or different EWGs such as COOH or nitro and/or one or more identical EDGs such as OH or methoxy, or both, using mild NaCNBH₃ in weak acidic media (1% acetic acid in methanol), for 2 hours at room temperature. In this way, the Compounds 1-9 were obtained in yields within the range of 58%-92% and the known compound 9-acridinylamino-acetic acid was obtained in 91% yield.

In another embodiment of the invention, the method consists in nucleophilic aromatic substitution (herein S_{N}Ar) reaction, a substitution reaction in which the nucleophile displaces a good leaving group, such as a halide, on an aromatic ring. This method is suitable for the preparation of compounds of formula I wherein X is aryl substituted with at least one EWG and optionally further substituted with one EDG or X is heteroaryl unsubstituted or substituted with one or more identical or different EWGs, one EDG, or both. In the context of exploring the rapid derivatization of the 9-aminoacridine scaffold, it was surprisingly found that the amino (NH₂) group at position 9 is nucleophilic enough to undergo nucleophilic aromatic substitution to give 9-aminoacridines derivatives such as 9-anilinoacridines. In the S_{N}Ar reaction, electron withdrawing groups activate the ring towards nucleophilic attack, for example, if there are nitro functional groups positioned ortho or para to the halide leaving group. Scheme 2 hereinafter depicts several different S_{N}Ar reactions. In one embodiment, 9-aminoacridine was reacted with a phenyl halide substituted at position ortho by an EWG group represented by R₄ and unsubstituted or substituted at position para by an EWG group represented by R₅, thus obtaining the Compound 10 (R₄═NO₂, R₅═H), Compound 12 (R₄═NO₂, R₅═NO₂), Compound 13 (R₄═NO₂, R₅═COOH), Compound 15 (R₄═CF₃, R₅═COOMe), Compound 16 (R₄═NO₂, R₅═CN), and Compound 17 (R₄═NO₂, R₅═SO₃H), using commercially available synthons.

In another embodiment in Scheme 2,9-aminoacridine was reacted with 1,5-difluoro-2,4-dinitrobenzene, thus obtaining Compound 18.

In a further embodiment in Scheme 2,9-aminoacridine was reacted with 2-chloro-pyridine substituted at position 3 with the EWG group NO₂ and further substituted with an EWG or EDG represented by R₆, wherein R₆ is CH₃, Cl, CN or CO₂H. In this way, the derivatives Compound 24 (R₆═CH₃), Compound 25 (R₆═Cl); Compound 26 (R₆═CN); and Compound 27 (R₆═CO₂H), are obtained using commercially available synthons.

In yet another embodiment in Scheme 2,9-aminoacridine was reacted with 2-chloro-pyrimidine substituted with an EWG or EDG represented by R₆, wherein R₆ is CH₃, Cl, CN or CO₂H. In this way, the derivatives Compound 30 (R₆═CH₃), Compound 31 (R₆═Cl), Compound 32 (R₆═CN), Compound 33 (R₆═CO₂H), were obtained using commercially available synthons.

Scheme 3 depicts the reaction of 9-aminoacridine with 1-fluoro-2-methoxy-4-nitro-benzene thus obtaining the Compound 20 containing one EWG (NO₂) at position para and one EDG (OMe) at position ortho.

The S_{N}Ar reaction of the invention can be performed using any suitable base, for example, Cs₂ CO₃, NaOH, K₂ CO₃, t-BuONa and organic bases like diisopropyl amine, preferably Cs₂ CO₃, and in any suitable solvent, for example DMF, NMP and DMSO. In some embodiments, the S_{N}Ar reaction is performed in the presence of Cs₂ CO₃ in DMSO.

In another embodiment of the invention, the method consists in addition-elimination (AE) reaction for reacting 9-aminoacridines with polysubstituted haloquinones. This method is depicted in Scheme 4 hereinafter and is appropriate for preparing the compounds of formula I wherein X is benzoquinone or a polycyclic aromatic quinone. In some embodiments, represented by the letter “a” under the arrows, the reaction is conducted under ethanol reflux overnight. Thus, in one embodiment, 9-aminoacridine was reacted in boiling ethanol with a 2,3-dihalo aromatic polycyclic quinone unsubstituted or substituted in the phenyl ring by one or more EDGs represented by R₇. For example, by reaction of 9-aminoacridine with 2,3-dichloro-1,4-naphthoquinone or with 2,3-dichloro-6,7-dimethyl-1,4-naphthoquinone, the derivatives Compound 39 (R₇═H) or Compound 40 (R₇═CH₃), respectively, are obtained. In another embodiment, 9-aminoacridine was reacted in boiling ethanol with 2,3-dichloro-5,8-dihydroxynaphthalene-1,4-dione, to give Compound 41. In a further embodiment, 9-aminoacridine was reacted with benzoquinone substituted with one to four EWGs represented by X₁, such as Cl or Br, thus obtaining Compound 36 (X₁═X₁═Br); Compound 34 (X₁═X₁═Cl), and Compound 37 (X₁═Cl; X₁═H). The ethoxy groups at position 5 of the benzoquinone ring of Compounds 34, 36, 37 or at position 3 of the naphthoquinone ring the Compound 41 result from an additional AE reaction with the solvent (ethanol).

Scheme 4 further depicts the reaction of 9-aminoacridine with 2,3,5,6-tetrabromobenzoquinone under aprotic conditions (1 eq of Cs₂ CO₃ in DMF at 90° C. for 12 h), represented by the letter “b” under the arrows, thus obtaining the Compound 38. No ethoxy group is added under these conditions

Scheme 5 summarizes the Schemes 1 to 4 discussed above and depicts the three methods of “one-pot” synthetic approach used in the present invention: reductive amination, nucleophilic aromatic substitution (S_{N}Ar) and addition-elimination (AE) reaction that afford access to much larger scope of 9-aminoacridine derivatives.

It has also been found in accordance with the present invention that 9-aminoacridine derivatives of the invention containing strong EWGs such as NO₂ alone or together with COOH, or CF₃ together with CN or an amino acid residue of the formulas I and II can be prepared by solid phase synthesis using various functionalized resins such as, but not limited to, 2-chlorotrityl (Cl-Trt) resin (reactive Cl atom), Rink Amide-MBHA resin (reactive NH₂ group), hydroxylamine-Trt resin (reactive —ONH₂ group), Wang resin (reactive OH group), Polyamine-Trt resin (reactive polyamine —NH—(CH₂)ₙ—NH₂ or —NH—(CH₂)ₙ—N(Boc)-(CH₂)ₙ—NH₂ group), and Cysteamine-Trt resin (reactive —S—(CH₂)ₙ—NH₂ group).

Using solid phase synthesis it is easier to remove excess reactant or byproducts from the end product. When 9-aminoacridine derivatives containing amino acid residues are desired, the amino acid residue present in the molecule is first protected at all reactive functional groups. Any suitable protecting groups can be used such as Fmoc, Boc, Pbf, tbutyl, or any combination thereof. The two functional groups that are able to participate in the desired reaction between amino acids in the solution and on the resin can be controlled by the order of deprotection.

In one embodiment, compounds of Formula I containing an amino acid residue are prepared by solid phase synthesis using Rink Amide-MBHA resin. First, the Rink Amide-MBHA resin is reacted with the properly protected amino acid. After removal of the protecting group, for example, removal of Fmoc by reaction with 20% piperidine in NMP, the resin is reacted with a preactivated solution of 3-nitro-4-fluorobenzoic acid that attaches to the resin via the free amino group of the amino acid, followed by reaction with 9 aminoacridine. Cleavage of the resin is carried out with TFA, thus releasing the 9-anilinoacridine amino acid derivative with a CONH₂ group. Scheme 6 depicts this procedure for the preparation of Compound 45 in high yield (97%) and purity (90%). The Rink Amide-MBHA resin was loaded with protected arginine (Fmoc-(L)Arg(Pbf)-OH). The Fmoc was removed from the resin-(L)Arg(Pbf)-OH molecule which was then reacted with preactivated 3-nitro-4-fluorobenzoic acid, followed by reaction with 9-aminoacridine under nucleophilic aromatic substitution conditions.

In another embodiment, compounds of Formula I containing an amino acid residue are prepared by solid phase synthesis using Cl-Trt resin similarly to the method using Rink Amide-MBHA resin. The loading and cleavage conditions may be different. The 9-anilinoacridine amino acid derivative is obtained with a free COOH group.

For preparation of derivatives of Formula I not containing an amino acid residue, the Cl-Trt or Rink Amide-MBHA resin is reacted with a preactivated solution of 4-fluoro-3-nitrobenzoic acid that attaches to the resin. The obtained resin is washed and then reacted with 9-aminoacridine under nucleophilic aromatic substitution conditions with Cs₂ CO₃ in DMF. Cleavage of the resin gives the 9-aminoacridine derivative in form of amide (with Rink Amide-MBHA resin) or in form of free COOH (with Cl-Trt resin). As an example, Scheme 7 depicts the preparation of Compound 13 by solid phase synthesis using the Cl-Trt resin in high yield (93%) and purity (94%). and of Compound 14 by solid phase synthesis using the Rink Amide-MBHA resin high yield (93%) and purity (94%).

As mentioned above, the solid phase synthesis is appropriate for the preparation of 9-aminoacridine derivatives of the invention containing strong EWGs such as NO₂ alone or together with NO₂ or COOH, or CF₃ together with CN or COOMe. Thus, if 3-nitro-4-fluorobenzoic acid is replaced by another suitable compound with such substituents, then other derivatives of the invention can be obtained. For example, by reaction with activated 1-nitro-4-fluorobenzene, the Compound 11 is obtained; by reaction with activated 1,3-dinitro-4-fluorobenzene, the Compound 12 is obtained; by reaction with activated 3-nitro-4-fluorobenzonitrile, the Compound 16 is obtained; by reaction with activated 1,3-dinitro-4,6-difluorobenzene, the Compound 18 is obtained; by reaction with activated 3-fluoromethyl-4-fluorobenzoic acid methyl ester, the Compound 15 is obtained.

Other 9-aminoacridine derivatives of the invention of Formulas I and II containing amino acid residues can be obtained by solid phase synthesis using the Cl-Trt or Rink Amide-MBHA amide.

Scheme 8 illustrates the solid phase synthesis in which Fmoc-(L)Lys(Boc)-OH protected lysine was loaded to the Rink Amide-MBHA and the Boc-protected lysine attached to the resin was coupled to Compound 13. After cleavage of the resin, Compound 46 (with CONH₂) was obtained in high yield (87%). The same scheme depicts the solid phase synthesis of the derivative of Formula II, Compound 47, when the resin was Cl-Trt and the Fmoc-(L)Lys(Fmoc)-OH protected lysine was loaded on the Cl-Trt resin and the unprotected lysine attached to the resin was coupled to Compound 13. After cleavage of the resin, Compound 47 (with free COOH) was obtained in high yield (84%).

Scheme 9 illustrates the solid phase synthesis of derivatives of the invention of Formula Ia containing a serine, glycine, arginine or lysine residue (Compounds 43, 44, 45, 46, respectively) in which FmocGly-OH, Fmoc(L)Ser(tBu)-OH, Fmoc(L)Lys(Boc)-OH or Fmoc(L)Arg(Pbf)-OH were loaded to the Rink Amide-MBHA, and the monoprotected amino acid —NH-AA(PG=protecting group)-H was reacted with preactivated 3-nitro-4-fluorobenzoic acid as described above. When the protected amino acids Fmoc(L)Lys(Fmoc)-OH, Fmoc(L)Orn(Fmoc)-OH, or Fmoc(L)DAB(Fmoc)-OH were loaded to the Rink Amide-MBHA, and the unprotected amino acid —NH-(L)Lys(NH₂)—H attached to the resin was reacted with preactivated 3-nitro-4-fluorobenzoic acid as described above, then the derivatives of Formula II Compounds 48, 49, 50 were obtained.

Scheme 10 illustrates the solid phase synthesis of the bis-anilinoacridine-MBPP conjugate Compound 51. Initially, the peptide of SEQ ID NO:1 was synthesized on a Rink Amide-MBHA resin as described in Example 14 hereinafter. Coupling of Fmoc-βAla-OH to the peptide of SEQ ID NO:1 afforded the peptide of SEQ ID NO:2. Then, coupling of the Fmoc(L)Lys(Fmoc)-OH was performed, preactivated 3-nitro-4-fluorobenzoic acid was added to the resin and reaction with 9-aminoacridine under nucleophilic aromatic substitution conditions was conducted.

The solid phase synthesis of the compounds of the invention affords a new approach to novel medicinally-important mono- and bis-9-anilinoacridine derivatives as described. Such synthetic strategy rapidly generates 9-anilinoacridines with variable spacer lengths and charged, polar or hydrophobic residues at desired positions, which can increase binding affinity, conformation stability, intracellular transport and/or biological activity of the 9-anilinoacridine-based candidates for development as drugs.

Many antitumour agents, including the anthracyclines, epipodophyllotoxins and mitoxantrone target DNA topoisomerase II arresting cell proliferation. Important 9-anilinoacridine drugs, e.g., Amsacrine and 3-(9-acridinylamino)-5-hydroxymethyl-aniline (AHMA), and their derivatives also play an important role in medicine and are successful candidates for treatment of cancer, viral and prion diseases. These compounds have been assigned as powerful DNA intercalators inhibiting DNA replication by forming topoisomerase II-DNA complexes, causing DNA strand breakdown and subsequent apoptosis. Apparently, the known “traditional” solution syntheses of 9-anilinoacridine analogs involve several steps in harsh conditions, requiring laborious purification of intermediates and final compounds. Evidently, solid phase organic synthesis (SPOS) is a fast and efficient experimental technique that has converged to create a productive approach to the discovery of bioactive hits.

Halobenzoquinones and halonaphthoquinones are widely used as anticancer agents with bioreductive properties. Members of this class of compounds can be selectively activated to cytotoxic species by reduction. The selective bioactivation may be due to elevated levels of some reductases in certain tumors, or to hypoxia. Apparently, bioreductive drugs are more toxic to more acidic hypoxic cells than to well oxygenated ones. According to the present invention, we designed a strategy to combine a bioreductive quinone moiety with a DNA-intercalating 9-aminoacridine, in order to obtain novel bifunctional bioreductive anticancer compounds. As far as the inventors are aware, such bifunctional bioreductive anticancer compounds using the DNA-intercalator as component have not been reported in the literature. The rationale behind the idea was to use the DNA-intercalator 9-aminoacridine for locating the drug between the DNA strands and creating active species by reductive metabolism in aerobic conditions to cause additional damage.

According to the present invention, the compounds can be in the form of pharmaceutically acceptable salts.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylamino-ethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound used in the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It is to be understood that, as used herein, references to the 9-aminoacridine derivatives herein in the application are meant to also include the pharmaceutically acceptable salts thereof.

The compounds of the present invention exhibit cytotoxic activity and are potential candidates for use as anticancer agents.

The present invention also provides pharmaceutical compositions comprising a 9-aminoacridine derivative of the invention or a pharmaceutically acceptable salt thereof as the active ingredient and a pharmaceutically acceptable carrier.

The pharmaceutical compositions of the present invention can be formulated for administration by a variety of routes including oral, rectal, transdermal, subcutaneous, intravenous, intramuscular, and intranasal. In some preferred embodiments, the compositions are administered systemically, in particular by injection, including infusion.

The pharmaceutical compositions are prepared in a manner well known in the pharmaceutical art and comprise the active ingredient along with an excipient or a carrier.

During the preparation, the active ingredient is usually mixed with an excipient or carrier or diluted by an excipient. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols, soft and hard gelatin capsules, suppositories, sterile injectable solutions, and sterile packaged powders.

In preparing a formulation, it may be necessary to mill the active ingredient to provide the appropriate particle size prior to combining with the other ingredients. If the active compound is substantially insoluble, it ordinarily is milled to a particle size of less than 200 mesh. If the active ingredient is substantially water soluble, the particle size is normally adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methylcellulose. The formulations can additionally include lubricating agents such as talc, magnesium stearate, and mineral oil; wetting agents; emulsifying and suspending agents; preserving agents such as methyl- and propylhydroxybenzoates; sweetening agents; and flavoring agents. The compositions can be formulated so as to provide quick, sustained or delayed release of the active ingredient after administration to the patient by employing procedures known in the art.

The compositions are preferably formulated in a unit dosage form, each dosage containing from about 0.1 to about 500 mg. The term “unit dosage forms” refers to physically discrete units suitable as unitary dosages for human subjects and other mammals, each unit containing a predetermined quantity of the active compound calculated to produce the desired therapeutic effect, in association with a suitable pharmaceutical excipient.

The active ingredient is effective over a wide dosage range and is generally administered in a therapeutically effective amount. It will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings; such materials include a number of polymeric acids and mixtures of polymeric acids with materials such as shellac, cetyl alcohol, and cellulose acetate.

The liquid forms in which the compositions of the present invention may be incorporated, for administration orally or by injection, include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Direct or indirect placement techniques may be used when it is desirable or necessary to introduce the pharmaceutical composition to the brain. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 incorporated herein by reference as if fully set forth. Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions, which can transiently open the blood-brain barrier.

In some embodiments, the compounds and compositions of the invention are useful for treating cancer. The 9-aminoacridine derivatives of the invention are DNA-intercalating agents. They play an important role due to their antiproliferative properties based on inhibition of both DNA topoisomerases I and II. Besides, they have also another mechanism of action, as they suppress PI3K/AKT/mTOR, p53 and NF-kB pathways that are frequently deregulated in tumor cells. The ability to simultaneously affect several biological pathways makes them a prototype of a previously uncharacterized class of bitargeted anticancer drugs.

In addition, since the 9-aminoacridines of the invention are fluorescent, they can also be used as fluorescent probes to detect cancer cells.

Thus, the compounds of the invention can be used as therapeutic and diagnostic agents, for example for treatment and detection of several types of cancer such as, but not limited to, renal, breast, colon, melanoma, ovarian, prostate, brain, skin, lung, esophagus and bladder cancers. In some embodiments, the compounds are for treatment of renal, ovarian and breast cancer.

When a 9-aminoacridine conjugate with a tumor-specific peptide is used, the peptide that has high affinity to specific tumor cells will preferentially target the 9-aminoacridine moiety to the tumor or treated site. In this context, also polypeptides such as tumor-specific antibodies can be conjugated to the 9-aminoacridine moiety and used according to the invention.

The compounds of the invention can be used alone for treatment of cancer or along with other anticancer agents.

The invention will now be illustrated by the following Examples which, together with the above description, illustrate some embodiments of the invention in a non-limiting fashion.

### EXAMPLES

The following abbreviations are used herein: Boc: t-butyloxycarbonyl; DCM: dichloromethane; DMF: dimethylformamide; DMSO: dimethyl sulfoxide; NMM: N-methylmorpholine; THF: tetrahydrofuran; TFA: trifluoroacetic acid; PE: petrol ether; PyBOP: benzotriazol-1-yl-oxytripyrrolidinophosphonium hexafluorophosphate.

### EXPERIMENTAL

Coupling reactions were performed under nitrogen atmosphere and in a commercial acid- and base-free DMF (99.8% Aldrich) or dry dichloromethane, AR (Aldrich). Most of the reactions were carried out in the dark. THF was dried over sodium and distilled prior to use. All other solvents and reagents were pure grade and used without purification, as well as the following commercially available chemicals: 9-aminoacridine (Merck), amino acids, resins and coupling reagents (Chem-Impex Int), building blocks and synthones (Acros). Reactions were monitored by thin layer chromatography (TLC) performed on silica gel sheets containing UV fluorescent indicator (60 F254 Merck). Chromatography was carried out by standard flash chromatography on silica gel 60. ¹H and ¹³C NMR spectra were recorded on Bruker AC 200, Bruker AC 400, and Bruker AC 700 spectrometers (200, 400, and 700 MHz for ¹H, respectively, and 50, 100, and 175 MHz for ¹³C, 2D COSY, TOCSY, NOESY, ROESY, HMBC, and HMQC spectroscopy). Chemical shifts, δ, are reported in ppm taking residual CHCl₃ or DMSO-d₆ as the reference. All chemical shifts are reported with respect to tetramethylsilane (TMS). Mass spectra were measured in the positive and negative modes using a quadrupole mass spectrometer equipped with an electro spray ionization source and cross-flow inlet. Analytical high-pressure liquid chromatography (HPLC) was performed on a 250×4.2 mm Lichroprep RP-18 column from Merck, with a 1 mL/min flow and detection at 214 nm. The eluents were triply distilled water and HPLC-grade CH₃ CN containing 0.1% TFA or MeOH. The concentration of all the samples was 0.5%. SPE was performed on LiChrospher 60 RP-18 columns purchased from Agilent Technologies.

### Example 1

### Synthesis of N-benzyl-9-aminoacridines Compounds 1-9 via Reductive Amination

The reaction is depicted in Scheme 1 with glyoxylic acid as the aliphatic aldehyde and benzaldehyde substituted with 1 to 3 groups selected from EWGs or EDGs as the aromatic aldehyde.

1.1 General procedure

9-Aminoacridine (0.194 g, 1 mmol) and corresponding aldehyde (1 mmol) were added to 5 mL of MeOH/AcOH (99:1) and stirred at room temperature until starting materials dissolved (15 min). Then, NaCNBH₃ (0.09 g, 1.5 mmol) in small portions was added with stirring. After additional stirring for 3 h at room temperature, the solvent was evaporated and the residue was taken into acetone. The precipitate was filtered in vacuum, washed with acetone and dried to give a product as yellow solid. Compounds that did not precipitate were purified by flash chromatography on silica gel 60 (5% MeOH in ethyl acetate) to yield pure yellow products.

In this way, the 9-benzylaminoacridine derivatives of Formula I described below were obtained in good yields ranging from 58% to 92% by reacting 9-aminoacridine with the corresponding (hetero)aromatic aldehyde. Such high yields are not achieved using the classical “reverse” synthetic approach in which 9-chloroacridine is reacted with benzyl amines.

Compound 1:

2-(acridin-9-ylaminomethyl)benzoic acid, by reaction with 2-formylbenzoic acid (yield—92%);

Compound 2:

4-(acridin-9-ylaminomethyl)benzoic acid, by reaction with 4-formylbenzoic acid (yield—89%);

Compound 3:

2-((acridin-9-ylamino)methyl)-4-nitrophenol, by reaction with 2-hydroxy-5-nitro-benzaldehyde (yield—87%);

Compound 4:

N-(2-nitro-5-hydroxy-benzyl)acridin-9-amine, by reaction with 2-nitro-5-hydroxy-benzaldehyde;

Compound 5:

N-(2,4,6-trimethyl-benzyl)acridin-9-amine, by reaction with 2,4,6-trimethyl-benzaldehyde (yield—66%, after chromatographic purification);

Compound 6:

N-(3,4,5-trimethoxybenzyl)acridin-9-amine, by reaction with 3,4,5-trimethoxy-benzaldehyde (yield—68%, after chromatographic purification);

Compound 7:

N-(2-bromo-4,5-dimethoxy-benzyl)acridin-9-amine, by reaction with 2-bromo-4,5-dimethoxy-benzaldehyde (yield—72%, after chromatographic purification);

Compound 8:

N-(2-hydroxy-naphthylmethyl)acridin-9-amine, by reaction with 2-hydroxy-naphthaldehyde (yield—83%); and

Compound 9:

N-(5-methoxy-indol-3-ylmethyl)acridin-9-amine, by reaction with 5-methoxy-indole-3-carboxaldehyde (yield—58%, after chromatographic purification).

For comparison, the compound 9-acridinylamino-acetic acid was obtained in 91% yield by reacting 9-aminoacridine with oxalic acid semialdehyde (COOH—CHO).

### Example 2

### Synthesis of 2-(acridin-9-ylaminomethyl)benzoic acid—Compound 1

9-Aminoacridine (0.194 g, 1 mmol) and 2-formylbenzoic acid (0.15 g, 1 mmol) were added to 5 mL of MeOH/AcOH (99:1) and stirred at room temperature until starting material dissolved (15 min). Then, NaCNBH₃ (0.09 g, 1.5 mmol) in small portions was added with stirring. After additional stirring for 3 h at room temperature, the solvent was evaporated and the residue was taken into acetone. The precipitate was filtered in vacuum, washed with acetone and dried to give 0.3 g of pure product as yellow solid (0.3 g, 92% yield): FT-IR (νₘₐₓ, KBr): 3500-3180 (bs), 1700 (C═O), 1640, 1290 cm⁻¹; HRMS (DI, m/z) calculated for C₂₁H₁₆N₂O₂ (MH⁺) 329.364. found 329.363. ¹H NMR (δ, ppm, CDCl₃): 8.50 (d, 2H, J=7.00 Hz), 7.92 (d, 2H, J=7.00 Hz), 7.77 (d, 1H, J=6.80 Hz), 7.68-7.65 (m, 2H), 7.39-7.35 (m, 3H), 4.54 (s, 2H, —NH—CH₂—); ¹³C NMR (δ, ppm, CDCl₃): 171.9 (CO₂H), 168.7, 152.7, 146.5, 141.5, 139.0, 131.5, 130.6, 130.1, 128.6, 128.2, 126.3, 125.5, 123.6, 122.1, 64.1.

### Example 3

### Synthesis of 2-((acridin-9-ylamino)methyl)-4-nitrophenol—Compound 3

9-Aminoacridine (0.194 g, 1 mmol) and 2-hydroxy-5-nitrobenzaldehyde (0.152 g, 1 mmol) were added to 5 mL of MeOH/AcOH (99:1) and stirred at room temperature until starting material dissolved (15 min). Then, NaCNBH₃ (0.09 g, 1.5 mmol) in small portions was added with stirring. After additional stirring for 3 h at room temperature, the solvent was evaporated and the residue was taken into acetone. The precipitate was filtered in vacuum, washed with acetone and dried to give 0.29 g of pure product as yellow solid (87% yield), FT-IR (νₘₐₓ, KBr): 3400-3150(bs), 1620, 1350, 1190 cm⁻¹; HRMS (DI, m/z) calcd for C₂₀H₁₅N₃O₃ (MH⁺) 346.111. found 346.115. ¹H NMR (DMSO-d₆): δ 8.42 (d, 2H, J=7.00 Hz), 7.95 (dd, 1H, J=7.00, 1.80 Hz), 7.84-7.80 (m, 3H), 7.69-7.65 (m, 2H)), 7.34-7.31 (m, 2H), 6.66 (d, 1H, J=6.80 Hz), 4.68 (s, 2H, —NH—CH₂—), ¹³C NMR (δ, ppm, DMSO-d₆): 163.8, 137.0, 130.5, 130.0, 129.5. 126.3, 125.9, 125.5, 124.8, 124.1, 123.6, 123.0, 122.1, 121.6, 112.5, 64.1.

### Example 4

### Synthesis of N-(3,4,5-trimethoxybenzyl)acridin-9-amine—Compound 6

9-Aminoacridine (0.194 g, 1 mmol) and 3,4,5-trimethoxybenzaldehyde (0.196 g, 1 mmol) were added to 5 mL of MeOH/AcOH (99:1) and stirred at room temperature until starting material dissolved (15 min). Then, NaCNBH₃ (0.09 g, 1.5 mmol) in small portions was added with stirring. After additional stirring for 3 h at room temperature, the solvent was evaporated and the residue was purified by flash chromatography on silica gel 60 (5% MeOH in ethyl acetate) to yield pure yellow product. 0.25 g of pure product as yellow solid (68% yield): HRMS (DI, m/z) calculated for C₂₂H₂₃N₂O₃ (MH⁺) 375.163. found 375.161. ¹H NMR (δ, ppm, DMSO-d₆): 8.45 (d, 2H, J=7.00 Hz), 7.88 (d, 2H, J=7.00 Hz), 7.72 (t, 2H, d, 2H, J=7.00 Hz), 7.55 (t, 2H, J=7.00 Hz), 6.68 (s, 2H), 4.57 (s, 2H, —NH—CH₂—), 3.76 (s, 3H), 3.74 (s, 6H). ¹³C NMR (δ, ppm, DMSO-d₆): 162.2, 141.0, 131.5, 129.0, 128.5, 126.2, 125.7, 125.5, 124.6, 124.3, 123.4, 122.9, 122.4, 121.7, 114.5, 66.1, 60.7, 58.3.

### Example 5

### Synthesis of 9-anilino-acridines via S_{N}Ar

The nucleophilic aromatic substitution reaction of 9-aminoacridine with a haloaryl or haloheteroaryl compound is depicted in Scheme 2. Representative electrophilic haloaryls bearing one or two strong EWGs such as NO₂, COOH, COOMe, CN, CF₃ and SO₃H, were reacted with 9-aminoacridine in presence of 1 equivalent (half molar ratio) of Cs₂ CO₃ in heated DMF (90° C.) for 12 h, yielding the 9-anilinoacridine derivatives Compounds 10-18 in good yields. A characteristic of some embodiments of the invention is formation of an anilino tether in 9-aminoacridine with two EW groups, what is very difficult to obtain using the standard “reverse” approach, namely nucleophilic substitution of deactivated anilines on 9-chloroacridines. The anilinic amine in such a “reverse” reaction is strongly deactivated by EW groups leading mostly to unreacted materials or black tar.

An additional advantage of the S_{N}Ar reaction of 9-aminoacridines is the commercial availability of appropriately substituted haloaryls. Interestingly, 4-chloro-3-nitrobenzoic acid, which bears acidic CO₂H, smoothly undergoes the S_{N}Ar to give Compound 13 even in presence of basic Cs₂ CO₃. One would expect a possible suppression of electrophilic potential of the acid by formed carboxyl anion, what was surprisingly not observed, most probably due to more favorable neutralization of released chloride by Cs₂ CO₃. Moreover, the introduced CO₂H group in Compound 13 by S_{N}Ar as well as in carboxy and hydroxy groups in previously mentioned Compounds 1, 2, 3, and 8 can serve as a linking group to various carriers for possible delivery of the 9-aminoacridine-based drugs.

An attempt was made to obtain bis-9-aminoacridine by reacting 2.5 equivalents of 9-aminoacridine with one equivalent of 1,5-difluoro-2,4-dinitrobenzene, but only the mono adduct Compound 18 was obtained in moderate yield, most probably due to the severe steric hindrance.

The 9-aminoacridine derivatives described herein that possess acidic CO₂H or phenol groups, namely Compounds 1, 2, 3, 8, and 13, precipitated from acetone as pure (more than 94% purity by HPLC) yellow solids, while unreacted starting materials and solvents were completely soluble in acetone. Such a phenomenon can be attributed to formation of poorly soluble (in organic solvents) zwitterions from the basic pyridinium amine and the acidic proton, what significantly simplifies the isolation process. This hypothesis is supported by the observed low-field shift for anilinic H-2′, ortho to EW positively-charged 9-aminoacridinium moiety (7.23, J=6.5 Hz) in the ¹H NMR spectrum. Usually protons ortho to strong electron donating (ED) free amine base are high-field shifted around 6.5 ppm.

### 5.1 General S_{N}Ar Procedure

9-Aminoacridine (0.194 g, 1 mmol), corresponding haloaryl (1 mmol) and Cs₂ CO₃ (0.161 g, 0.5 mmol) were heated in 5 mL of dry DMF at 90° C. for 12 h. While heating, the color of the reaction mixture changed to dark red. After completion of the reaction (TLC monitoring in DCM) the reaction mixture was cooled and poured into water. In case of Compound 13 the pH was adjusted to 6 by careful addition of 0.1 N HCl. The precipitate was collected by filtration, washed several times with water and dried to give orange crude solid. The compounds (except Compound 13 that precipitates in pure form) were purified by flash chromatography on silica gel 60 (DCM), to yield pure products.

In this way, the 9-anilinoacridine derivatives below of Formula I were obtained in good yields ranging from 58% to 92% by reacting 9-aminoacridine with the corresponding (hetero)aromatic haloaldehyde. Such high yields are not achieved using the classical “reverse” synthetic approach in which 9-chloroacridine is reacted with aniline derivatives.

Compound 10:

N-(2-nitrophenyl)acridin-9-amine, by reaction with 1-bromo-2-nitrobenzene (yield—73%, after chromatographic purification);

Compound 11:

N-(3-nitrophenyl)acridin-9-amine, by reaction with 1-bromo-3-nitrobenzene;

Compound 12:

N-(2,4-dinitrophenyl)acridin-9-amine, by reaction with 1-chloro-2,4-dinitrobenzene (yield—78%, after chromatographic purification);

Compound 13:

4-(acridin-9-ylamino)-3-nitrobenzoic acid, by reaction with 4-chloro-3-nitrobenzoic acid (yield—86%);

Compound 14:

4-(acridin-9-ylamino)-3-nitrobenzamide, by reaction with 4-chloro-3-nitrobenzamide;

Compound 15:

4-(acridin-9-ylamino)-3-trifluoromethyl-benzoic acid methyl ester, by reaction with 4-fluoro-3-trifluoromethyl-benzoic acid methyl ester (yield—76%, after chromatographic purification);

Compound 16:

4-(acridin-9-ylamino)-3-nitro-benzonitrile, by reaction with 4-chloro-3-nitro-benzonitrile (yield—71%, after chromatographic purification);

Compound 17:

4-(acridin-9-ylamino)-3-nitro-benzenesulfonic acid, by reaction with 4-chloro-3-nitro-benzenesulfonic acid;

Compound 18:

N-(2,4-dinitro-5-fluorophenyl)acridin-9-amine, by reaction with 1,3-difluoro-4,6-dinitrobenzene (yield—52%, after chromatographic purification);

Compound 19:

5-(acridin-9-ylamino)-2-nitrophenol, by reaction with 3-nitrophenol; and

Compound 20:

N-(2-methoxy-4-nitrophenyl)acridin-9-amine, by reaction with 1-fluoro-2-methoxy-4-nitrobenzene.

### 5.2 Characterization of 5-(acridin-9-ylamino)-2-nitrophenol (Compound 19)

Orange solid after chromatography (10% MeOH/CHCl₃), 0.19 g, 58% yield; R_{f}=0.50 (MeOH/CHCl₃, 1:9), HRMS (CI, m/z) calculated for C₁₉H₁₃N₃O₃ (MH⁺) 332.0957. found 332.1056. ¹H NMR (300 MHz, DMSO-d6): 10.08 (bs, OH), 8.38-8.27 (m, 3H), 8.02 (d, 2H, J=6.8 Hz), 7.88 (t, 2H, J=6.8 Hz), 7.61 (t, 2H, J=6.8 Hz), 6.83 (d, 2H, J=6.8 Hz), 6.24 (bs, NH), ¹³C NMR (75 MHz, DMSO-d6): 152.5, 142.2, 141.0, 137.6, 135.2, 130.3, 127.7, 125.2, 124.6, 123.2, 121.8, 119.5, 118.2.

### 5.3 Characterization of N-(2-methoxy-4-nitrophenyl)acridin-9-amine (Compound 20)

Orange solid after chromatography (5% MeOH/CHCl₃), 0.21 g, 64% yield; R_{f}=0.60 (MeOH/CHCl₃, 5:95), HRMS (CI, m/z) calculated for C₂₀H₁₅N₃O₃ (MH⁺) 346.1113. found 346.1774. ¹H NMR (300 MHz, DMSO-d6): 8.37 (d, 2H, J=6.8 Hz), 8.08 (d, 2H, J=6.8 Hz), 7.90-7.76 (m, 2H), 7.70-7.65 (m, 1H), 7.53-7.49 (m, 2H), 7.30-7,20 (m, 2H), 4.18 (s, OMe). ¹³C NMR (75 MHz, DMSO-d6): 153.3, 145.7, 143.1, 134.4, 131.0, 128.3, 127.2, 126.0, 124.8, 124.0, 122.4, 121.7, 120.2, 56.5.

### Example 6

### Synthesis of N-(2-nitrophenyl)acridin-9-amine—Compound 10

9-Aminoacridine (0.194 g, 1 mmol), 1-bromo-2-nitrobenzene (0.201 g, 1 mmol) and Cs₂ CO₃ (0.161 g, 0.5 mmol) were heated in 5 mL of dry DMF at 90° C. for 12 h. While heating, the color of the reaction mixture changed to dark red. After completion of the reaction (TLC monitoring in DCM) the reaction mixture was cooled and poured into water. The precipitate was collected by filtration, washed several times with water and dried to give orange crude solid. The crude product was purified by flash chromatography on silica gel 60 (DCM) to yield pure orange Compound 10 (0.23 g, 73% yield): HRMS (DI, m/z) calculated for C₁₉H₁₃N₃O₂ (MH⁺) 316.325. found 316.321. ¹H NMR (δ, ppm, CDCl₃): 9.32. (bs, 1, NH), 8.24 (d, 2H, J=6.8 Hz), 8.01-7.88 (m, 3H), 7.71-7.53 (m, 4H), 7.33 (t, 1H, J=7.0 Hz), 6.62 (d, 1H, J=7.0 Hz). ¹³C NMR (δ, ppm, CDCl₃): 165.2, 154.3, 147.7, 144.1, 133.4, 130.0, 129.7, 128.7, 127.2, 125.6, 124.7, 122.3, 120.5.

### Example 7

### Synthesis of 4-(acridin-9-ylamino)-3-nitrobenzoic acid—Compound 13

9-Aminoacridine (0.194 g, 1 mmol), 4-chloro-3-nitrobenzoic acid (0.20 g, 1 mmol) and Cs₂ CO₃ (0.161 g, 0.5 mmol) were heated in 5 mL of dry DMF at 90° C. for 12 h. While heating, the color of the reaction mixture changed to dark red. After completion of the reaction (TLC monitoring in 10% MeOH/DCM) the reaction mixture was cooled and poured into water. The pH was adjusted to 6 by careful addition of 0.1 N HCl. The precipitate was collected by filtration, washed several times with water and dried to give pure Compound 13 as orange crude solid (0.335 g, 86% yield): νₘₐₓ (KBr): 3450-3200(bs), 1705 (C═O), 1600, 1245 cm⁻¹; HRMS (DI, m/z) calculated for C₂₀H₁₃N₃O₄ (MH⁺) 360.091. found 360.101. ¹H NMR (δ, ppm, DMSO-d₆): 8.56. (s, 1H), 8.30-8.27 (m, 3H), 8.00-7.90 (m, 3H), 7.66-7.58 (m, 3H), 7.17 (d, 1H, J=6.80 Hz); ¹³C NMR (6, ppm, DMSO-d₆): 173.0 (CO₂H), 164.2, 153.1, 148.2, 143.5, 132.4, 131.0, 128.9, 127.7, 127.0, 126.2, 125.6, 121.4, 120.5.

### Example 8

### Synthesis of 2-pyridyl- and 2-pyrimidyl-9-aminoacridines—Compounds 21-33

The heteroaromatic pyridine and pyrimidine Compounds 21-33 were synthesized by nucleophilic aromatic substitution as described in Example 2 above by reaction of 9-aminoacridine with the suitable chloropyridine or chloropyrimidine in the presence of one equivalent of Cs₂ CO₃ in DMF at 90° C. The following compounds were obtained in good yields:

Compound 21:

N-(3-nitropyrid-2-yl)acridin-9-amine, by reaction with 2-chloro-3-nitropyridine (yield—81%);

Compound 22:

N-(pyrid-2-yl)acridin-9-amine, by reaction with 2-chloro-pyridine (yield—47%);

Compound 23:

6-(acridin-9-ylamino)nicotinonitrile, by reaction with 6-chloro-nicotinonitrile (yield—68%);

Compound 24:

N-(5-methyl-3-nitropyridin-2-yl)acridin-9-amine, by reaction with 2-chloro-5-methyl-3-nitropyridine;

Compound 25:

N-(5-chloro-3-nitropyridin-2-yl)acridin-9-amine, by reaction with 2,5-dichloro-3-nitropyridine;

Compound 26:

6-(acridin-9-ylamino)-5-nitronicotinonitrile, by reaction with 6-chloro-5-nitronicotinonitrile;

Compound 27:

6-(acridin-9-ylamino)-5-nitronicotinic acid, by reaction with 6-chloro-5-nitronicotinic acid;

Compound 28:

N-(pyrimid-2-yl)acridin-9-amine, by reaction with 2-chloro-pyrimidine (yield—75%); and

Compound 29:

N-(5-bromopyrimid-2-yl)acridin-9-amine, by reaction with 2-chloro-5-bromopyrimidine (yield—83%).

Compound 30:

N-(5-methylpyrimidin-2-yl)acridin-9-amine, by reaction with 2-chloro-5-methylpyrimidine or 2-bromo-5-methylpyrimidine;

Compound 31:

N-(5-chloropyrimidin-2-yl)acridin-9-amine, by reaction with 2,5 dichloropyrimidine;

Compound 32:

2-(acridin-9-ylamino)pyrimidine-5-carbonitrile, by reaction with 2-chloropyrimidine-5-carbonitrile;

Compound 33:

2-(acridin-9-ylamino)pyrimidine-5-carboxylic acid, by reaction with 2-chloropyrimidine-5-carboxylic acid.

### 8.1 Characterization of N-(pyrid-2-yl)acridin-9-amine (Compound 22)

Orange solid after chromatography (CHCl₃), 0.13 g, 47% yield; R_{f}=0.65 (EtOAc), HRMS (CI, m/z) calculated for C₁₈H₁₃N₃ (MH⁺) 272.111. found 272.086. ¹H NMR (300 MHz, DMSO-d6): 9.12 (br s, 1, NH), 8.23 (d, 2H, J=6.8 Hz), 8.04-7.89 (m, 3H), 7.76-7.51 (m, 3H), 7.38-7.27 (m, 2H), 6.74-6.68 (m, 2H). ¹³C NMR (75 MHz, DMSO-d6): 157.1, 150.6, 148.2, 141.5, 130.3, 129.0, 125.2, 122.6, 121.0, 119.6, 113.2, 109.5.

### 8.2 Characterization of 6-(acridin-9-ylamino)nicotinonitrile (Compound 23)

Orange solid after chromatography (CHCl₃), 0.18 g, 68% yield; R_{f}=0.60 (EtOAc), HRMS (CI, m/z) calculated for C₁₉H₁₂N₄ (MH⁺) 297.1062. found 297.1114; FT-IR (νₘₐₓ, KBr): 2250, 1570, 1430, 1105 cm⁻¹. ¹H NMR (300 MHz, DMSO-d6): 9.85 (br s, 1, NH), 8.34-8.11 (m, 3H), 8.10-7.97 (m, 2H), 7.82-7.57 (m, 2H), 7.39-7.18 (m, 4H); ¹³C NMR (75 MHz, DMSO-d6): 160.2, 151.3, 149.4, 143.5, 141.7, 130.0, 127.1, 125.2, 121.0, 118.2, 116.1, 114.5, 103.9.

### 8.3 Characterization of N-(3-nitropyrid-2-yl)acridin-9-amine (Compound 21)

Reddish solid after chromatography (CHCl₃), 0.25 g, 81% yield; R_{f}=0.50 (EtOAc), HRMS (CI, m/z) calculated for C₁₈H₁₂N₄O₂ (MH⁺) 317.0960. found 317.1205. ¹H NMR (300 MHz, DMSO-d6): 11.52 (br s, 1, NH), 8.60-8.41 (m, 2H), 8.22-8.03 (m, 2H), 7.87-7.41 (m, 5H), 7.19-7.04 (m, 2H). ¹³C NMR (75 MHz, DMSO-d6): 154.3, 152.0, 150.4, 143.2, 136.6, 134.3, 130.2, 128.9, 127.7, 120.8, 115.3, 113.5.

### 8.4 Characterization of N-(pyrimid-2-yl)acridin-9-amine (Compound 28)

Orange solid after chromatography (CHCl₃), 0.19 g, 75% yield; R_{f}=0.40 (EtOAc), HRMS (CI, m/z) calculated for C₁₇H₁₂N₄ (MH⁺) 273.1062. found 273.1222. ¹H NMR (300 MHz, DMSO-d6): 9.80 (br s, 1, NH), 8.52 (d, 2H, J=6.7 Hz), 8.28 (d, 2H, J=6.8 Hz), 8.12-7.98 (m, 4H), 7.64-7.38 (m, 2H), 7.08 (t, 1H, J=6.7 Hz). ¹³C NMR (75 MHz, DMSO-d6): 171.3, 156.1, 150.0, 148.3, 144.4, 135.7, 130.3, 129.8, 124.2, 121.7, 117.1.

### 8.5 Characterization of N-(5-bromopyrimid-2-yl)acridin-9-amine (Compound 29)

Orange solid after chromatography (CHCl₃), 0.24 g, 83% yield; R=0.70 (MeOH/CHCl₃, 2:98), HRMS (CI, m/z) calcd for C₁₇H₁₁BrN₄ (MH⁺) 351.0167. found 350.0198 (46%), 352.0208 (43%); ¹H NMR (300 MHz, DMSO-d6): 9.64 (br s, 1, NH), 8.58 (s, 2H), 8.24 (d, 2H, J=6.8 Hz), 8.10-7.92 (m, 4H), 7.60-7.33 (m, 2H), ¹³C NMR (75 MHz, DMSO-d6): 154.7, 151.2, 143.4, 142.9, 133.7, 131.1, 127.2, 120.0, 119.8, 116.1, 114.4.

### Example 9

### Synthesis of 9-aminoacridine derivatives containing substituted quinone radicals [Compounds 34-41] via addition-elimination (AE) or S_{N}Ar reaction

Two one-pot reaction modes were employed: (i) addition-elimination reaction by refluxing in ethanol overnight, and (ii) S_{N}Ar reaction with Cs₂ CO₃ in DMF at 90° C. for 12 h. The successful one pot synthesis of the end products resulted in moderate to good yields.

### 9.1 General procedure for the synthesis via addition-elimination (AE) reaction

9-Aminoacridine (0.194 g, 1 mmol) and haloquinone compound (1 mmol) were refluxed in 15 mL of EtOH for overnight. While heating, the color of the reaction mixture in most cases changed to dark red or gray. After completion of the reaction (TLC monitoring in CH₂ Cl₂) the mixture was cooled and evaporated to give a crude red or gray solid. The products were purified by flash column chromatography on silica gel 60 to yield corresponding products.

### 9.2 General procedure for the synthesis via S_{N}Ar reaction

9-Aminoacridine (0.194 g, 1 mmol), haloaryl or haloquinone compound (1 mmol), and Cs₂ CO₃ (0.161 g, 0.5 mmol) were heated in 5 mL of dry DMF at 90° C. for 12 h. While heating, the color of the reaction mixture in most cases changed to dark red. After completion of the reaction (TLC monitoring in 5% MeOH in CH₂ Cl₂) the mixture was cooled and poured into water. The resulting precipitate was collected by filtration, washed several times with water, and dried to give a crude red or orange solid. The products were purified by flash column chromatography on silica gel 60 to yield the corresponding products.

The following compounds were obtained, most of them in moderate to good yields after chromatography:

Compound 34:

2-(acridin-9-ylamino)-3-ethoxy,5,6-dichlorobenzoquinone, by reaction with tetrachlorobenzoquinone in ethanol under reflux overnight (yield—57%);

Compound 35:

2-(acridin-9-ylamino)-3-bromo-5-ethoxy-benzoquinone, by reaction with tetrabromobenzoquinone in ethanol under reflux overnight (yield—3%);

Compound 36:

2-(acridin-9-ylamino)-3,6-dibromo-5-ethoxycyclohexa-2,5-diene-1,4-dione, by reaction with tetrabromobenzoquinone in ethanol under reflux overnight;

Compound 37:

2-(acridin-9-ylamino)-3-chloro-5-ethoxycyclohexa-2,5-diene-1,4-dione;

Compound 38:

2-(acridin-9-ylamino)-3,5,6-tribromobenzoquinone, by reaction with tetrabromobenzoquinone and Cs₂ CO₃ in dry DMF at 90° C. for 12 h. (yield—79%);

Compound 39:

2-(acridin-9-ylamino)-3-chloronaphthoquinone, by reaction with 2,3-dichloronaphthoquinone and Cs₂ CO₃ in dry DMF at 90° C. for 12 h. (yield—82%);

Compound 40:

2-(acridin-9-ylamino)-3-chloro-6,7-dimethylnaphthoquinone, by reaction with 2,3-dichloro-6,7-dimethylnaphthoquinone and Cs₂ CO₃ in dry DMF at 90° C. for 12 h. (yield—88%); and

Compound 41:

2-(acridin-9-ylamino)-3-ethoxy-5,8-dihydroxy-naphthoquinone, by reaction with 2,3-dichloro-5,8-dihydroxy-naphthoquinone in ethanol under reflux overnight (yield—86%);

To demonstrate the synthetic potential of AE reaction with 9-aminoacridine, we employed two classes of representative quinones: halobenzoquinones and halonaphthoquinones yielding respective bifunctional quinono-9-aminoacridine derivatives 26-31 under mild conditions. During the examination, we noticed that different reaction conditions led to different products. When an equimolar amount of tetrachlorobenzoquinone was reacted with 9-aminoacridine in refluxing ethanol overnight, the product 2-(acridin-9-ylamino)-3,6-dichloro-5-ethoxycyclohexa-2,5-diene-1,4-dione-(Compound 34) was obtained, after chromatography (silica gel, CHCl₃), in 57% yield. Interestingly, this molecule possesses one ethoxyl group in E position to 9-amino of acridine as a result of additional AE reaction with EtOH (chemical structure was characterized by d-NOE, HMBC, HMQC experiments). The same conditions used for the reaction of analogous tetrabromobenzoquinone led to the formation of single reduced E product 2-(acridin-9-ylamino)-3-bromo-5-ethoxy-benzoquinone (Compound 35) in very low yield (3%). The MS spectrum (C₂₁H₁₅ BrN₂O₃, m/z (M⁻) 421.22 (92%), 424.28 (100%) clearly showed the presence of only one bromine atom and ¹H NMR confirmed the presence of one quinone hydrogen (5.93 s). On the other hand, using aprotic conditions (1 eq of Cs₂ CO₃ in DMF at 90° C. for 12 h) only 2,3,5,6-tetrabromobenzoquinone underwent classical S_{N}Ar reaction leading to 2-(acridin-9-ylamino)-3,5,6-tribromobenzoquinone (Compound 38) in 79% yield after purification. Other tetrahaloquinones, 2,3,5,6-tetrachloro- and 2,3,5,6-tetrafluorobenzoquinone, under these conditions afforded unidentified mixtures. Moving forward, we decided to switch at that point to chlorobenzoquinone reactants for obtaining more massive benzoquinone moiety on 9-aminoacridine by AE reaction. Benzoquinone, for example, can bear additional amino, hydroxyl and methoxy groups that, together with quinonic the ketone group, are able to enhance biologically important chelating properties, leading to formation of DNA damaging reactive species.

2,3-Dichloro or 2,3-dichloro-6,7-dimethyl-naphthalene-1,4-dione was reacted with 9-aminoacridine in boiling ethanol affording gray crude products (39 and 40, respectively). Simple work up followed by evaporation of solvent and subsequent purification by flash chromatography (silica gel, EtOAc:PE, 1:2) gave corresponding products 39 and 40 in 82% and 88% yields, respectively. The existence of Cl atom was confirmed by MS spectra clearly showing 3:1 of chlorine isotope ratio (C₂₃H₁₄ ClN₂O₂, m/z (MH⁺) 385.213 (98%), 387.324 (31%) for Compound 39, C₂₅H₁₈ClN₂O₂, m/z (MH⁺): 412.311 (92%), 414.298 (27%) for Compound 40).

Encouraged by this result, we reacted the biologically more interesting 2,3-dichloro-5,8-dihydroxynaphthalene-1,4-dione with 9-aminoacridine in boiling ethanol to afford a reddish crude Compound 41 that did not require any further purification and was analyzed as is. Surprisingly, the spectral analysis identified Compound 41 as a result of double EA reaction, once by 9-aminoacridine and secondly by EtOH, similarly to quinone derivatives 34 and 35 but oppositely to benzoquinone analogs 39 and 40. The structural conformation was supported by an observed high-field shift in the ¹H NMR spectrum for the nonsymmetrical phenolic protons at δ 11.82 and δ 12.33 and typical low-field shift for ethoxy group (δ 4.66, q, J=6.5 Hz for 2H and δ 1.37, t, J=6.5 Hz for 3H).

The following compounds were further characterized:

### 9.3 Characterization of 2-(acridin-9-ylamino)-3,6-dichloro-5-ethoxycyclohexa-2,5-diene-1,4-dione (Compound 34)

Greenish solid after chromatography (CHCl₃), 0.27 g, 57% yield; R_{f}=0.70 (CHCl₃), HRMS (ES, m/z) calculated for C₂₁H₁₄Cl₂N₂O₃ (M⁻) 413.2535. found 413.2703 (48%), 425.2756 (31%); 427.2732 (7%). ¹H NMR (300 MHz, DMSO-d6): 12.40 (bs, NH), 8.01 (d, J=6.8 Hz, 2H-4,5), 7.78 (t, J=6.8 Hz, 2H-2,7), 7.63 (d, J=7.2 Hz, 2H-1,8), 7.30 (t, J=7.2 Hz, 2H-3,6), 4.63 (q, J=5.8 Hz, O—CH₂), 1.36 (t, J=6.2 Hz, —CH₃). ¹³C NMR (75 MHz, DMSO-d6): 172.91 (s, C-1′(4′), C═O), 172.69 (s, C-4′(1′), C═O), 158.16 (s, C-9), 156.51 (s, C-5′), 149.43 (s, C-2′), 139.55 (s, 2C-4a,8b), 133.59 (d, 2C-2,7), 126.57 (d, 2C-4,5), 122.65 (d, 2C-3,6), 119.67 (s, C-3′), 117.54 (d, 2C-1,8), 116.39 (s, 2C-4-b,8a), 112.12 (s, C-6′), 70.70 (t, O—CH₂—), 15.88 (q, CH₃).

### 9.4 Characterization of 2-(acridin-9-ylamino)-3-bromo-5-ethoxycyclohexa-2,5-diene-1,4-dione (Compound 35)

Greenish solid after chromatography (CHCl₃), 0.01 g, 3% yield; R_{f}=0.60 (CHCl₃), HRMS (ES, m/z) calculated for C₂₁H₅BrN₂O₂(M⁻) 422.0188. found 421.2843 (89%), 423.2812 (100%); ¹H NMR (300 MHz, DMSO-d6): 12.28 (bs, NH), 7.96 (d, J=7.2 Hz, 2H-4,5), 7.75 (t, J=7.2 Hz, 2H-2,7), 7.60 (d, J=7.2 Hz, 2H-1,8), 7.29 (t, J=7.2 Hz, 2H-3,6), 5.92 (s, 1H-6′), 4.06 (q, J=6.2 Hz, O—CH₂—), 1.36 (t, J=6.2 Hz, —CH₃). ¹³C NMR (75 MHz, DMSO-d6): 178.05 (s, C-1′, C═O), 172.77 (s, C-4′, C═O), 160.16 (s, C-5′), 157.27 (s, C-9), 152.79 (s, C-2′), 139.59 (s, 2C-4a,8b), 133.40 (d, 2C-2,7), 126.43 (d, 2C-4,5), 122.51 (d, 2C-3,6), 117.42 (d, 2C-1,8), 116.45 (s, 2C-4b,8a), 104.91 (s, C-3′), 103.90 (s, C-6′), 65.56 (t, O—CH₂—), 13.74 (q, CH₃).

### 9.5 Characterization of 2-(acridin-9-ylamino)-3-chloronaphthalene-1,4-dione (Compound 39)

Dark gray solid after chromatography (CHCl₃), 0.34 g, 82% yield; R_{f}=0.75 (CHCl₃), HRMS (ES, m/z) calculated for C₂₃H₁₃ ClN₂O₂ (MH⁺) 385.0688. found 385.1365 (100%), 387.1410 (36%). ¹H NMR (300 MHz, DMSO-d6): 8.20-8.03 (m, 3H), 7.90-780 (m, 3H), 7.72-7.65 (m, 2H), 7.60-7.57 (m, 2H), 7.23 (t, 2H, J=6.9 Hz). ¹³C NMR (75 MHz, DMSO-d6): 181.3, 181.0, 164.1, 157.2, 148.4, 146.2, 138.9, 135.5, 132.8, 133.0, 132.5, 130.1, 129.3, 127.3, 125.7, 120.8, 118.2.

### 9.6 Characterization of 2-(acridin-9-ylamino)-3-chloro-6,7-dimethylnaphthalene-1,4-dione (Compound 40)

Dark gray solid after chromatography (CHCl₃), 0.38 g, 88% yield; R_{f}=0.80 (CHCl₃), HRMS (ES, m/z) calculated for C₂₅H₁₇ClN₂O₂ (MH⁺) 413.0979. found 413.1098 (98%), 415.1108 (34%). ¹H NMR (300 MHz, DMSO-d6): 8.17-8.00 (m, 3H), 7.86-7.80 (m, 2H), 7.77-7.60 (m, 3H), 7.26 (t, 2H, J=6.9 Hz). ¹³C NMR (75 MHz, DMSO-d6): 181.1, 180.6, 164.0, 157.2, 148.5, 147.3, 137.8, 134.3, 133.7, 133.4, 131.9, 131.1, 129.0, 128.2, 126.7, 121.8, 117.5, 19.2, 18.7.

### 9.7 Characterization of 2-(acridin-9-ylamino)-3-ethoxy-5,8-dihydroxynaphthalene-1,4-dione (Compound 41)

Dark red solid 0.41 g, 86% yield; R_{f}=0.30 (MeOH/CHCl₃, 1:99), HRMS (ES, m/z) calculated for C₂₅H₁₈N₂O₅ (MH⁺) 427.1216. found 427.1344 (38%), 425.1308 (M-H, 100%). ¹H NMR (300 MHz, DMSO-d6): 12.10 (s, 1H, OH), 11.82 (s, 1H, OH), 10.16 (bs, 2H, NH₂⁺), 8.72 (d, J=7.0 Hz, 2H-1,8) 8.02-7.95 (m, 4H-3,4,5,6), 7.59 (t, 2H, J=7.0 Hz, 2H-2,7). 7.40-7.38 (m, 2H-6′,7′), 4.58 (q, J=6.2 Hz, —OCH₂—), 1.37 (t, J=6.2 Hz, —CH₃). ¹³C NMR (75 MHz, DMSO-d6): 181.56 (s, 2-C═O), 157.72 (s, C-9), 157.20 (s, C-3′), 156.88 (s, C-5′), 156.16 (s, C-8′), 139.25 (s, 2C-4a,8b), 135.48 (d, 2C-3,6), 129.52 (d, C-7′), 129.10 (d, C-6′), 127.38 (s, C-2′), 124.72 (d, 2C-1,8), 123.73 (d, 2C-2,7), 118.67 (d, 2C-4,5), 111.44 (s, 2C-4b,8a), 127.38 (s, C-2′), 111.24 (s, C-8b′), 110.46 (s, C-8a′), 70.67 (t, O—CH₂—), 15.83 (q, CH₃).

### 9.7 Characterization of 2-(acridin-9-ylamino)-3,5,6-tribromocyclohexa-2,5-diene-1,4-dione (Compound 38)

Dark green solid after chromatography (CHCl₃), 0.32 g, 79% yield; R_{f}=0.80 (CHCl₃), HRMS (CI, m/z) calculated for C₁₉H₉Br₃N₂O₂ (MH⁺) 534.8214. found 533.8017(35%), 534.8058 (100%), 535.8100 (98%), 536.8110 (27%); ¹H NMR (300 MHz, DMSO-d6): 8.07 (d, 2H, J=6.8 Hz), 7.81 (t, 2H, J=6.8 Hz), 7.54 (t, 2H, J=6.8 Hz), 6.92 (d, 2H, J=6.8 Hz). ¹³C NMR (75 MHz, DMSO-d6): 188.3, 181.9, 173.1, 168.4, 144.0, 140.8, 133.2, 131.7, 129.2, 126.5, 122.3, 121.6, 118.9.

### Example 10

### Preparation of Compounds 42-50 Containing Amino Acid Residue by Solid Phase Synthesis

### 10.1 General Procedure for Solid Phase Synthesis on Cl-Trt Resin:

To 2-chlorotrityl resin (0.2 g, 0.28 mmol loading) in a reactor was added a solution of properly protected amino acid (0.26 mmol) in dry DMF (3.5 mL) and after addition of diisopropylethylamine (DIEA, 185 mL, 1.04 mmol) the reaction mixture was shaken for 1.5 h. After completion of the loading, dry MeOH (1.5 mL) was poured into the reactor and shaking continued for an additional 20 min. The solvent was filtered out and the following washings were sequentially performed: 2×DCM:MeOH:DIEA (17:2:1), 2×DCM, 2×DMF, 2×DCM, 2×DCM:DMF (1:1) (3 mL each). The Fmoc protecting group was removed by reaction with 20% piperidine in N-methyl-2-pyrrolidone (NMP) (2×15 min, 5 mL each) and subsequent washing (2×DCM, 2×DMF, 5 mL each). Then, a preactivated solution of 3-nitro-4-fluorobenzoic acid (0.78 mmol acid, 0.78 mmol PyBoP, 2.34 mmol DIEA in 4.5 mL DMF) was added to the resin and shaken for 2 h. Then the resin was washed with 2×DMF, 2×DCM (3 mL each) and the aromatic substitution procedure of 9-aminoacridine with 0.5 g Cs₂ CO₃ in 3 ml DMF was performed for 24 h. After washings by 2×H₂O, 2×DMF, 2×MeOH, 2×DCM and 2×DMF (3 mL each) the resin was transferred to a vial for cleavage and a cold solution of 1% TFA in DCM (2 mL) was added. After shaking for 30 min, the solution was collected and the resin was washed several times with DCM (3 mL each). After combining the organic solutions, the solvent was evaporated first by N₂ stream and then in vacuum to give after the usual work up (fast purification by solid-phase extraction pack RP-18, first washed with water and then extracted with acetonitrile, 5 mL each) the 9-anilinoacridine-amino acid conjugate with a free COOH group.

### 10.2 General Procedure for Solid Phase Synthesis on Rink Amide MBHA Resin:

The procedure for the synthesis on Rink amide MBHA resin is identical to the synthesis on Cl-Trt resin except for the loading and the cleavage:

Loading:

The Fmoc protecting group from Rink amide was removed by reaction with 20% piperidine in NMP (2×15 min, 5 mL each) and subsequent washing (2×DCM, 2×DMF, 5 mL each). Then, a preactivated solution of 3-nitro-4-fluorobenzoic acid (0.78 mmol acid, 0.78 mmol PyBoP, 2.34 mmol DIEA in 4.5 mL DMF) was added to the resin and shaken for 2 h. Then the resin was washed with 2×DMF, 2×DCM (3 mL each).

Cleavage:

The resin was transferred to a vial for cleavage and a cold solution of 2.5% H₂O/2.5% triisopropyl silane in 95% TFA (2 mL) was added. After shaking for 1.5 h, the solution was collected and the resin was washed with cold TFA (2×1 mL each). After combining the TFA solutions, the solvent was evaporated first by N₂ stream and then in vacuum to give after the usual work up (fast purification by solid-phase extraction pack RP-18, first washed with water and then extracted with acetonitrile, 5 mL each) the 9-anilinoacridine-amino acid conjugate with a CONH₂ group.

### Example 11

### Synthesis of 4-(acridin-9-ylamino)-N-(1-amino-5-guanidino-1-oxopentan-2-yl)-3-nitrobenzamide (Compound 45) by Solid Phase Synthesis

The synthesis was performed on Rink Amide MBHA as described in Example 10 and depicted in Scheme 6. To the resin was added a solution of the protected arginine molecule Fmoc-(L)Arg(Pbf)-OH. The resin-bound protected arginine was then reacted with 3-nitro-4-fluorobenzoic acid and after then with 9-aminoacridine and Cs₂ CO₃ in DMF for 24 h at room temperature. After cleavage of the resin, Compound 45 was obtained in good yield (97%) and 90% purity, and was characterized: as a yellowish powder (0.083 g, 97% yield). MS m/z 515.2 (MH⁺), ¹H NMR (300 MHz, DMSO-d₆): 8.80 (m, 1H), 8.63 (m, 1H), 8.92-8.88 (m, 2H), 8.50-8.35 (m, 4H), 7.8 (d, J=8 Hz, 2H), 7.60-7.45 (m, 4H), 7.14-6.95 (m, 3H), 4.21 (m, 1H), 3.26-3.02 (m, 4H), 1.55 (m, 2H).

### Example 12

### Solid Phase Synthesis of 9-Anilinoacridine Derivatives by S_{N}Ar

The solid phase synthesis of 2-nitro-4-carboxy- and 2-nitro-4-carboxamido-9-anilino derivatives is depicted in Scheme 7 herein. First, a solution of 3-nitro-4-fluorobenzoic acid in the solvents described in Scheme 7 was loaded on the Rink Amide MBHA or Cl-Trt resin and the resulting bound carboxamido or ester was reacted with 9-aminoacridine under S_{N}Ar conditions. After cleavage, from the Cl-Trt Resin the 2-nitro-4-carboxy-9-anilinoacridine (Compound 13) was obtained in high yield (93%) and purity (94%) and from the Rink Amide MBHA Resin the 2-nitro-4-carboxamido-9-anilinoacridine (Compound 14) was obtained in high yield (96%) and purity (93%).

### Example 13

### Solid Phase Synthesis Using Fmoc Protocol of Mono- and Bis-9-Anilino-Acridine Derivatives

The solid phase synthesis herein described is a synthetic strategy that rapidly generates 9-anilinoacridines with variable spacer lengths and charged, polar or hydrophobic residues at desired positions, which can increase binding affinity, conformation stability, intracellular transport and biological activity of the 9-aminoacridine-based drugs. The results described here can pave a way to more complicate conjugation with biomolecules such as peptides and proteins, modulating their activity, bioavailability and applicability.

As shown in the Scheme 8 above, we carried out the coupling of Compound 13 to preloaded Fmoc-(L)Lys(Boc)-OH on Rink amide MBHA and Cl-Trt resins. The purpose behind this experiment was to investigate the straightforward coupling of premade Compound 13 versus 9-anilinoacridine assembly approach to both resins. Thus, after applying standard Fmoc chemistry protocol (DIC/HOBt in DMF) the Compound 13 was successfully linked to α-amino deprotected lysine to afford after cleavage the corresponding Lys-9-anilinoacridine (Compound 46). When Fmoc-(L)Lys(Fmoc)-OH was used to load on Cl-Trt resin, corresponding bis-Lys(9-anilinoacridine)₂ (Compound 47) was obtained. Compounds 46 and 47 were synthesized in reasonable yields but insufficient for bioassay purity. At this stage we decided to move to “assembly” approach, as described in Example 10. Notably, the CO₂H on Compound 47 after cleavage from acid sensitive resin Cl-Trt resin can act as an anchor for conjugation chemistry. Noteworthy, the cleavage of Compound 47 was carried out in strong acidic conditions (TFA/H₂O/EDT (95:2.5:2.5)) as for Rink amide MBHA for solubility reasons.

The solid phase synthesis of other amino acid derivatives Compounds 43-46 is depicted in Scheme 9 below.

Thus it can be seen that high yields and high purity were obtained for derivatives obtained using protected glycine, serine, lysine and arginine as the amino acids.

Compound 42:

(2-(4-(acridin-9-ylamino)-3-nitrobenzamido)-3-hydroxypropanoic acid), (yield 64%);

Compound 43:

4-(acridin-9-ylamino)-N-(1-amino-3-hydroxy-1-oxoprop-2-yl)-3-nitrobenzamide, (yield 93%);

Compound 44:

4-(acridin-9-ylamino)-N-(2-amino-2-oxoethyl)-3-nitrobenzamide, (yield 84%);

Compound 45:

4-(acridin-9-ylamino)-N-(1-amino-5-guanidino-1-oxopent-2-yl)-3-nitro-benzamide (yield 97%);

Compound 46:

4-(acridin-9-ylamino)-N-(1,6-diamino-1-oxohex-2-yl)-3-nitro-benzamide (yield 87%);

Compound 47:

2,6-bis(4-(acridin-9-ylamino)-3-nitrobenzamido)hexanoic acid (yield 84%);

Compound 48:

N,N′-(6-amino-6-oxohexane-1,5-diyl)bis(4-(acridin-9-ylamino)-3-nitro-benzamide, (yield 94%);

Compound 49:

N,N′-(4-amino-4-oxobutane-13-diyl)bis(4-(acridin-9-ylamino)-3-nitrobenzamide), (yield 91%);

Compound 50:

N,N′-(5-amino-5-oxopentane-1,4-diyl)bis(4-(acridin-9-ylamino)-3-nitrobenzamide), yield 92%.

### Characterization of N,N′-(6-amino-6-oxohexane-1,5-diyl)bis(4-(acridin-9-ylamino)-3-nitrobenzamide) (Compound 48)

As a yellowish powder (0.137 g, 72% yield). MS m/z 828.4 (MH⁺), ¹H NMR (300 MHz, DMSO-d6): 8.83-8.65 (m, 4H), 8.50-8.35 (m, 4H), 8.18-8.02 (m, 2H), 7.83-7.79 (m, 2H), 7.67-7.55 (m, 3H), 7.54-7.46 (m, 3H), 7.20-6.85 (m, 6H), 4.40 (m, 1H), 3.25-3.07 (m, 4H), 1.89-1.80 (m, 2H), 1.58-1.40 (m, 4H).

### Characterization of (2,6-bis(4-(acridin-9-ylamino)-3-nitro benzamido)hexanoic acid) (Compound 47)

As a yellowish powder (0.122 g, 69% yield). MS m/z 829.8 (MH⁺), ¹H NMR (300 MHz, DMSO-d₆): 8.84-8.64 (m, 4H), 8.48-8.33 (m, 4H), 8.20-8.00 (m, 2H), 7.87-7.78 (m, 2H), 7.62-7.51 (m, 3H), 7.59-7.50 (m, 3H), 7.150-6.82 (m, 6H), 4.42 (m, 1H), 3.25-3.05 (m, 4H), 1.88-1.77 (m, 2H), 1.60-1.44 (m, 4H).

### Characterization of (2-(4-(acridin-9-ylamino)-3-nitrobenzamido)-3-hydroxypropanoic acid) (Compound 42)

As a yellow solid (0.105 g, 64% yield). MS m/z 447.1 (MH⁺), ¹H NMR (300 MHz, DMSO-d₆): 8.85-8.70 (m, 3H), 8.13 (d, J=8 Hz, 1H), 7.86 (d, J=8 Hz, 2H), 7.63 (t, J=8 Hz, 2H), 7.02 (d, J=8 Hz, 1H), 4.61 (m, 1H), 3.87 (d, J=6.5 Hz, 2H).

### Example 14

### Solid Phase Synthesis of bis-9-anilinoacridine-MBP Peptide Conjugate 51

The synthesis of the conjugate 51 is depicted in Scheme 10. Initially, the peptide MBP of SEQ ID NO:1 was synthesized on a Rink amide MBHA, using solid phase peptide synthesis (SPPS), by attaching to the resin protected amino acids according to the sequence SEQ ID NO:1. Then, the protected lysine residue Fmoc-L-Lys(Fmoc) was reacted with 3-nitro-4-fluorobenzoic acid, followed by the aromatic nucleophilic substitution procedure with 9-aminoacridine. More particularly, 4.5 g of Rink amide methylbenzhydrylamine (MBHA) resin (0.68 mmol/g) was swollen for 2 h in N-methylpyrrolidone (NMP) in a reaction vessel equipped with a sintered glass bottom, and placed on a shaker. The Fmoc group was removed with 20% piperidine in NMP (twice for min). After washing with NMP (five times for 2 min) and DCM (two times for 2 min), Fmoc removal was monitored using the ninhydrin Kaiser test. A coupling cycle was carried out with Fmoc-amino acids (3 eq.), bromo-tris pyrrolidino-phosphonium hexafluorophosphate (PyBrOP) (3 eq.), DIEA (6 eq.) in NMP for 2 h at room temperature. The resin was washed with NMP (five times for 2 min) and DCM (two times for 2 min). Reaction completion was monitored using the ninhydrin Kaiser test. Fmoc removal, washings and coupling of Fmoc-amino acids were performed as described above. Peptide elongation was performed by repeating the cycle described above. After the coupling of Fmoc-βAla-OH and subsequent Fmoc removal and washings as described above, coupling of the Fmoc-L-Lys(Fmoc)OH was performed with premixed amino acid acid (3-4.6 eq.), PyBrOP (3-4.6 eq.), and DIEA (6-9.2 eq.) in NMP. Reaction completion was monitored using the ninhydrin Kaiser test. Then, a preactivated solution of fluoronitrobenzoic acid (2.78 mmol acid, 2.78 mmol PyBoP, 7.34 mmol NMM in 10.5 mL DMF) was added to the resin and shaken for 40 min. Then the resin was washed with 2×DMF, 2×DCM (10 mL each) and the aromatic substitution procedure with 9-aminoacridine with 0.8 g Cs₂ CO₃ in 10 ml DMF was performed for 24 h. After washings with 2×H₂O, 2×DMF, 2×MeOH, 2×DCM and 2×DMF (7 mL each) the resin was transferred to a vial for cleavage and a cold solution of 2.5% H₂O/1.5% triisopropyl silane, 1% ethane dithiol in 95% TFA (5 mL) was added. After shaking for 1.5 h, the solution was collected and the resin was washed with cold TFA (2×1 mL each). After combining the TFA solutions, the solvent was evaporated first by N₂ stream and then in vacuum to give an oily crude peptide. The peptide was precipitated by treating the concentrated solution with cold ether (35 ml). The ether was removed by centrifugation and the precipitant was washed three times by cold ether. After the usual work up (fast purification by solid-phase extraction pack RP-18, first washed with water and then extracted with acetonitrile, 8 mL each), the peptide was collected and dried and submitted to purification by semipreparative HPLC (0.1% TFA/H₂O and CH₃ CN).

### Example 15

### In vitro Cytotoxicity of 9-aminoacridine Derivatives

### Protocol for Cytotoxicity:

Cells (250000/well) were cultured in 24-well plates for 24 h to 70-80% confluence or cells (20000/well) were cultured in 96-well plates for 24 h or overnight to 70-80% confluence. Four 24-well plates and one 96-well plate have been seeded. The following concentrations of the compounds were examined (each one in triplicate)-0 microgram/μl, 0.05 microgram/ml, 0.5 microgram/ml, 5 microgram/ml, 50 microgram/ml. The volume of wells in 24-well plates was 0.5 ml while that of 96-well plates was 0.1 ml. After the 24 h treatment the culture medium in the wells were replaced with 0.5 ml fresh medium containing 1:100 dilution of neutral red (10 microliters/1 ml). The plates were incubated for 2 h in a dark in culture incubator. The medium was aspirated and the cells washed twice with 0.5/0.1 ml of solution containing 1% CaCl₂ and 0.5% formaldehyde. The dye was extracted from cells upon addition of 0.5/0.1 ml 1% glacial acetic acid in 50% ethanol with 10 min incubation at room temperature. The absorbance was measured spectrophotometrically at a wavelength of 540 nm. The background absorbance measurement of the multiwell plates was done at 690 nm and subtract from 540 nm.

It has been previously demonstrated that antitumor 9-anilinoacridines including 3-(9-acridinylamino)-5-hydroxymethylanilines (AHMAs) and amsacrine are potent inhibitors of topoisomerase II and capable of intercalating into DNA doubled strands. Hence, they are suitable as a scaffold for constructing the new DNA-targeted compounds. Table 1 shows the cytotoxicity (IC₅₀ in micromolar) of some compounds of the invention against the cancer cell lines MDM-MD-A31 (renal cancer), MCF-7 (breast cancer), HT29 (colon carcinoma), OVCAR8 (ovarian cancer), NCI-ADR (associated with multidrug resistance (MDR) phenomena ovarian cancer), MCF-7mito (mitoxantrone selected and associated with MDR phenomena breast cancer) and H1299 (lung carcinoma), and comparison with commercial 9-aminoacridine and amsacrine drugs.

As shown in Table 1, the compounds of the present invention possess significant cytotoxicity with IC₅₀ values in submicromolar range. For instance, Compound 41 exhibits even greater activity against all above mentioned cell lines than the anticancer drug amsacrine.

TABLE 1
Broad spectrum of anticancer activity (IC₅₀ in μM)

| Cell line | MDM- | MCF7 | MCF7 | | | | |
|---|---|---|---|---|---|---|---|
| Compound | MD-A31 | wt | mito | OVCAR8 | NCIADR | H1299 | HT29 |
| 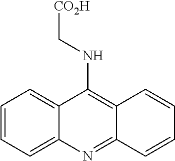 | 17.2 | 19.8 | 1.5 | 21.9 | 16.1 | 118.8 | 2.2 |
| 1 | 10.3 | 20.2 | 0.76 | 0.71 | 12.2 | 74.3 | 1.2 |
| 4 | 75.6 | 58.6 | 18.4 | 15.4 | 86.4 | >100 | 7.14 |
| 8 | 13.2 | 14.1 | 1.8 | 9.1 | 42.3 | 62.6 | 2.6 |
| 9-aminoacridine | 1.5 | 11.2 | 163.4 | 1.7 | 21.0 | 32.8 | 2.25 |
| 10 | >100 | >100 | 46.7 | >100 | 40.3 | >100 | 74.2 |
| 12 | >100 | 89.1 | 7.2 | 33.8 | 15.4 | >100 | 71.3 |
| 13 | 0.6 | 11.6 | 7.2 | 6.5 | 54.2 | 86.4 | 3.6 |
| 14 | 0.6 | — | 7.2 | | | | 3.6 |
| 18 | 0.3 | — | — | — | — | — | — |
| 20 | 0.1 | — | — | 0.6 | — | — | 1.2 |
| 21 | 0.8 | | 1.6 | — | — | 0.7 | — |
| 38 | — | — | — | 3.7 | — | — | — |
| 39 | 0.15 | | 0.8 | 1.2 | — | — | — |
| 41 | 0.06 | | 0.5 | — | — | 0.14 | 1.1 |
| 42 | 0.4 | — | 4.5 | — | — | — | 2.2 |
| 46 | 75.6 | 58.6 | 18.4 | 15.4 | 86.4 | >100 | 7.14 |

The preliminary results of biological activity and of structure-activity relationship (SAR) studies of the newly synthesized derivatives are presented in Table 1 above. It is shown herein that the Compounds 1 and 13 possessing EWG carboxylic group on the aniline tether were more cytotoxic than the corresponding 9-aminoacridine against all tested cell lines in vitro. In general, quinone moiety in 9-quinoneacridines did not increase the potency, while Compound 41 with two hydroxyls on the condensed benzo moiety was the most potent among the tested compounds, most probably due to enhancement of biologically important chelating properties, possibly leading to formation of more powerful DNA damaging reactive species.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims. For example, although the method was demonstrated using 9-aminoacridine as a starting material, some embodiments include substituted 9-aminoacridines, for example, substituted at any of the aromatic carbons of the acridine core.

Citation or identification of any reference in this application shall not be construed as an admission that such reference is available as prior art to the invention.

Section headings are used herein to ease understanding of the specification and should not be construed as necessarily limiting.

APPENDIX 1

| Compound | |
|---|---|
| Number | Structure |
| 1 | 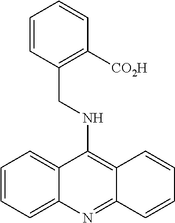 |
| 2 | 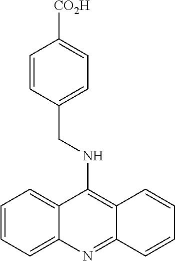 |
| 3 | 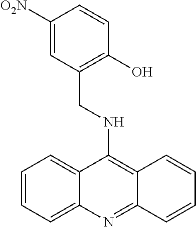 |
| 4 | 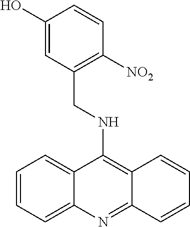 |
| 5 | 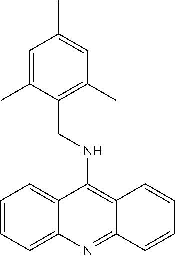 |
| 6 | 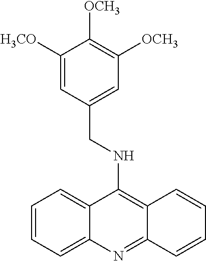 |
| 7 | 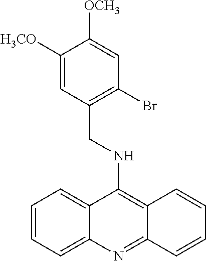 |
| 8 | 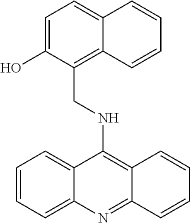 |
| 9 | 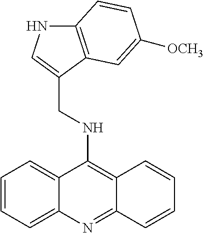 |
| 10 | 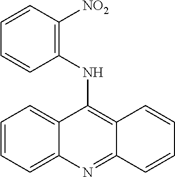 |
| 11 | 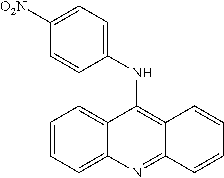 |
| 12 | 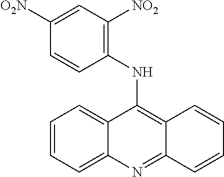 |
| 13 | 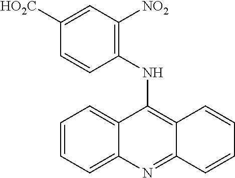 |
| 14 | 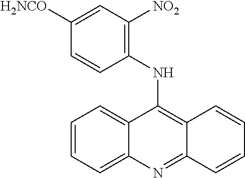 |
| 15 | 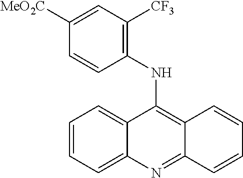 |
| 16 | 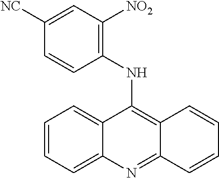 |
| 17 | 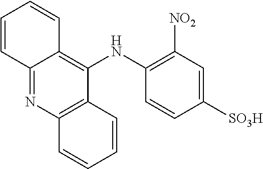 |
| 18 | 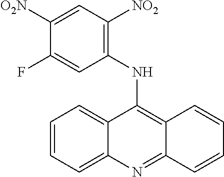 |
| 19 | 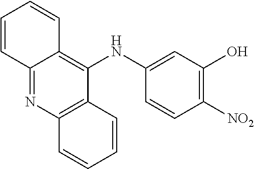 |
| 20 | 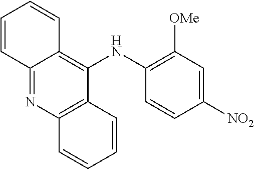 |
| 21 | 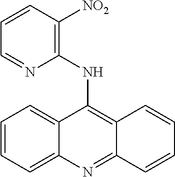 |
| 22 | 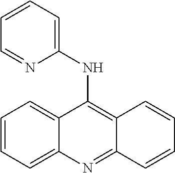 |
| 23 | 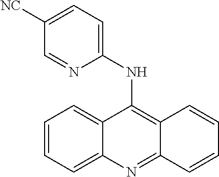 |
| 24 | 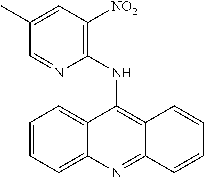 |
| 25 | 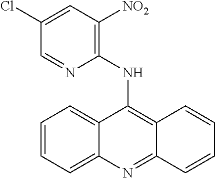 |
| 26 | 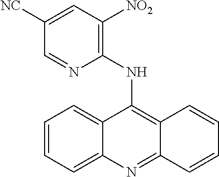 |
| 27 | 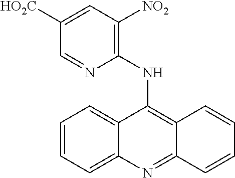 |
| 28 | 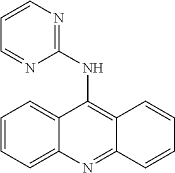 |
| 29 | 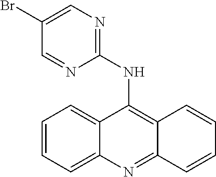 |
| 30 | 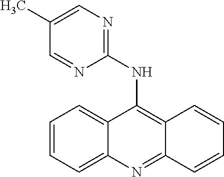 |
| 31 | 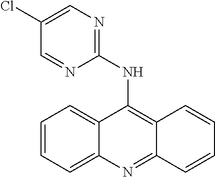 |
| 32 | 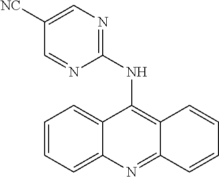 |
| 33 | 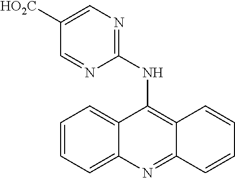 |
| 34 | 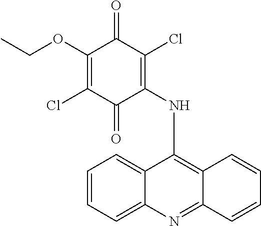 |
| 35 | 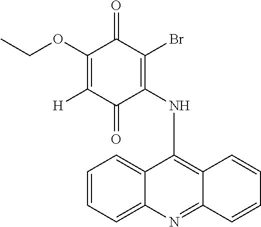 |
| 36 | 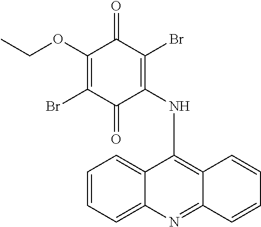 |
| 37 | 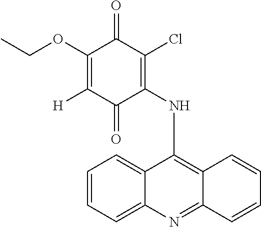 |
| 38 | 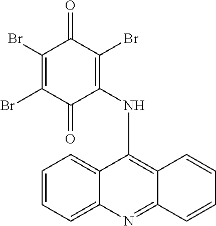 |
| 39 | 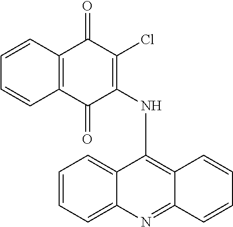 |
| 40 | 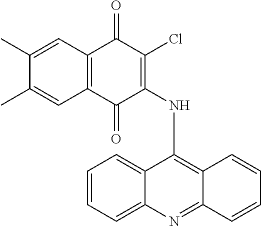 |
| 41 | 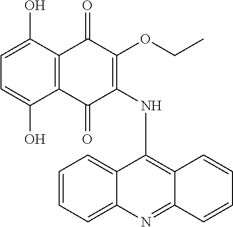 |
| 42 | 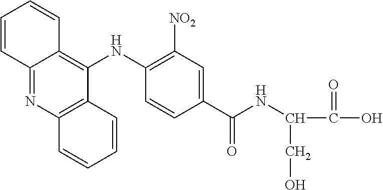 |
| 43 | 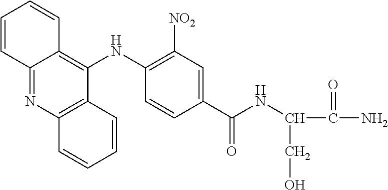 |
| 44 | 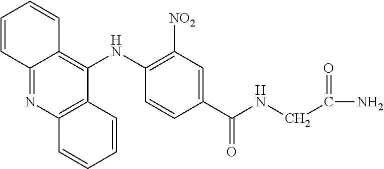 |
| 45 | 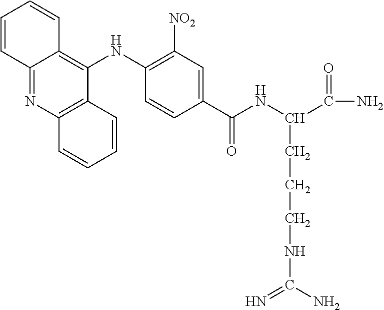 |
| 46 | 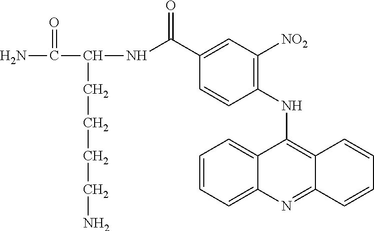 |
| 47 | 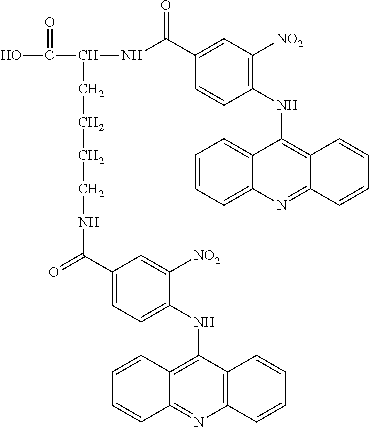 |
| 48 | 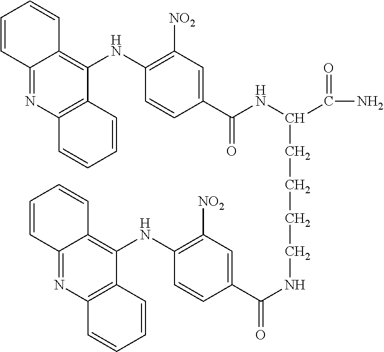 |
| 49 | 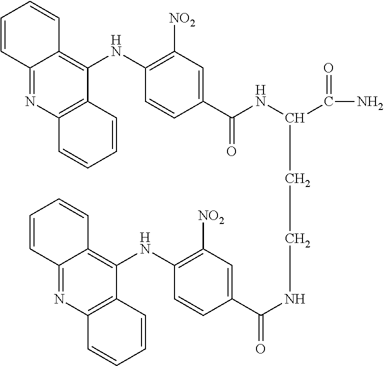 |
| 50 | 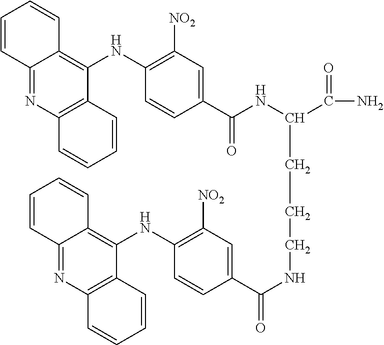 |
| 51 | 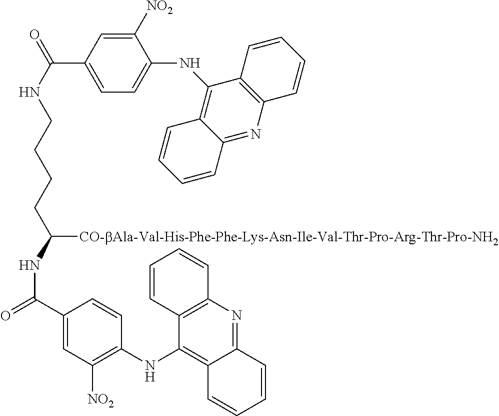 |

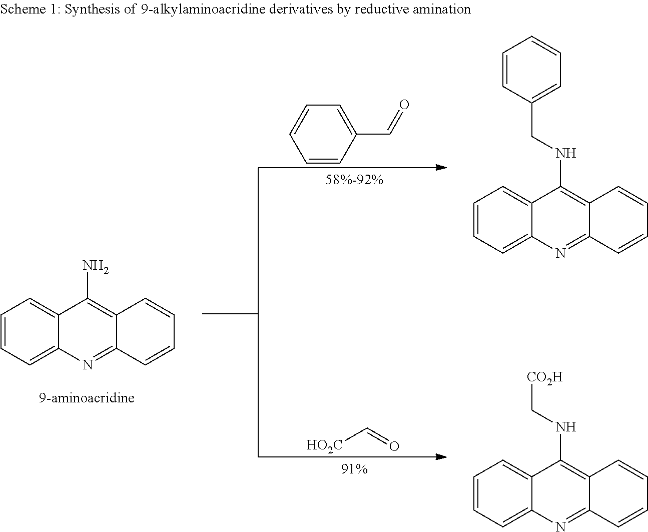

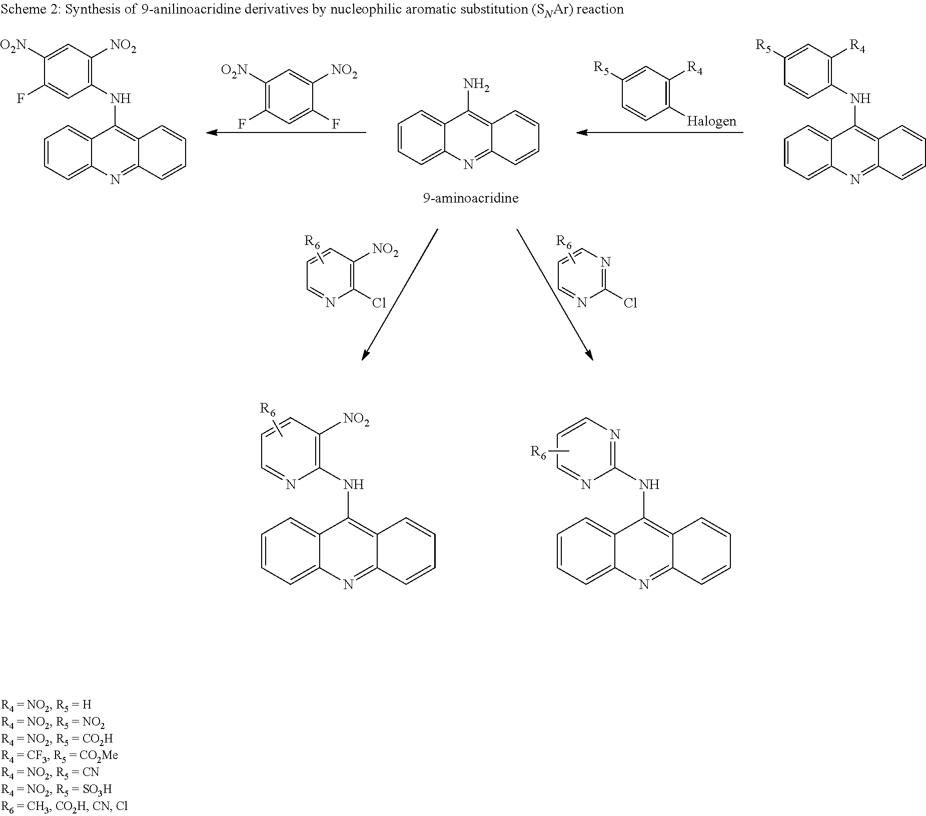

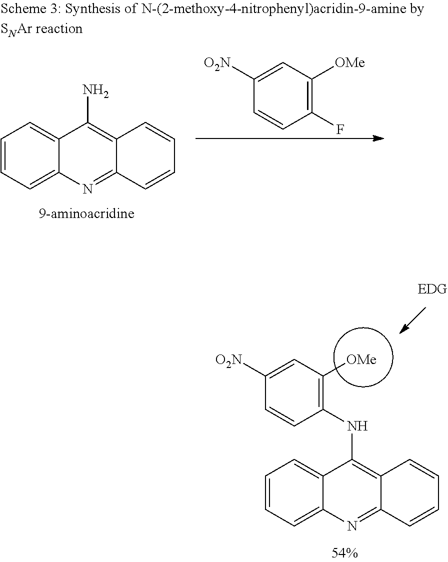

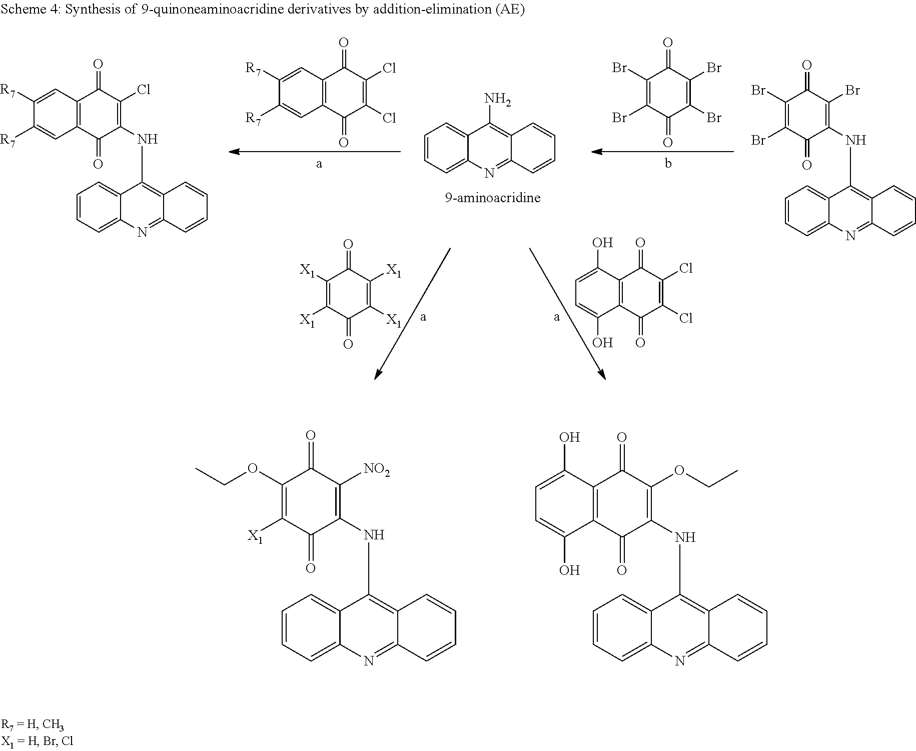

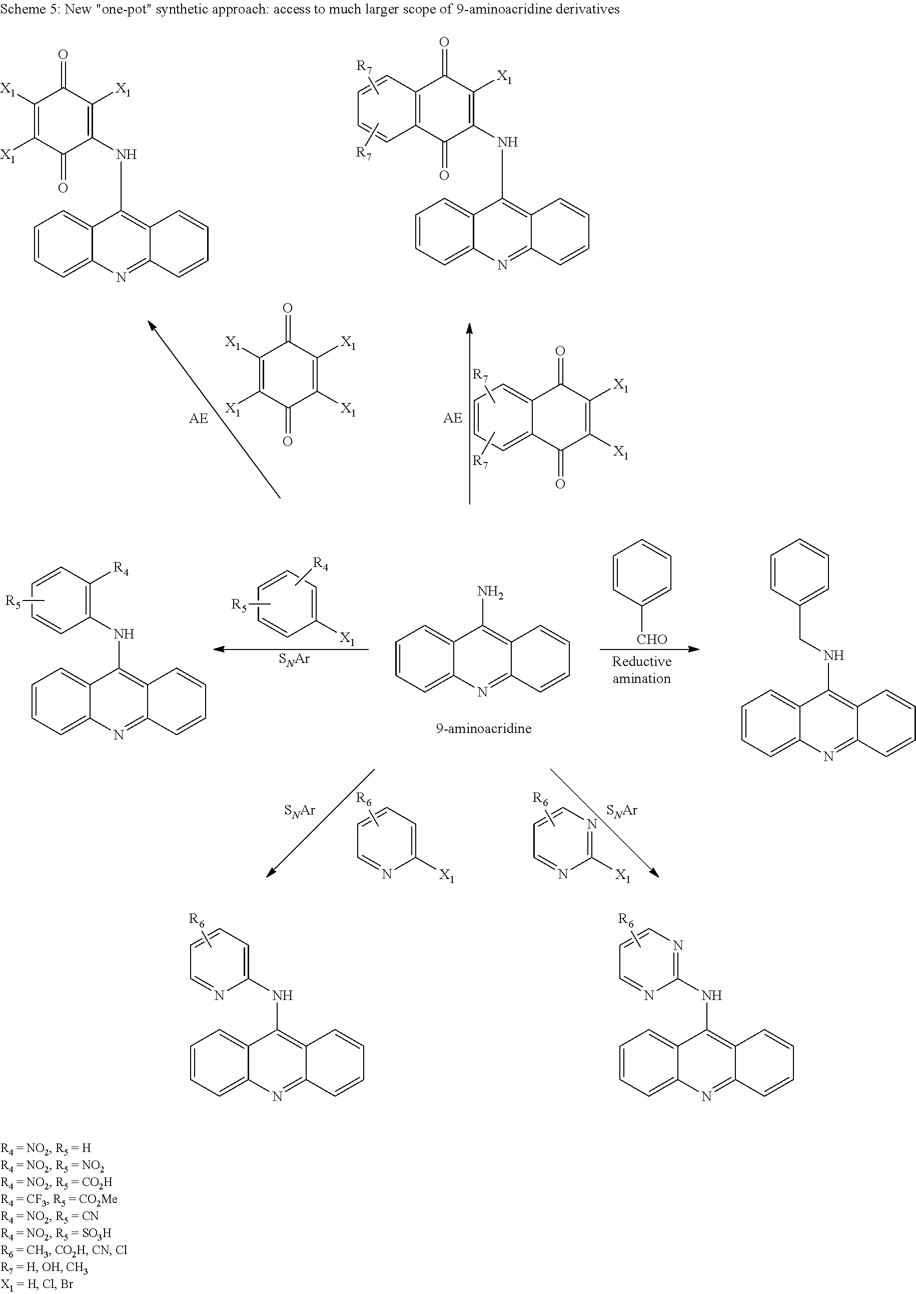

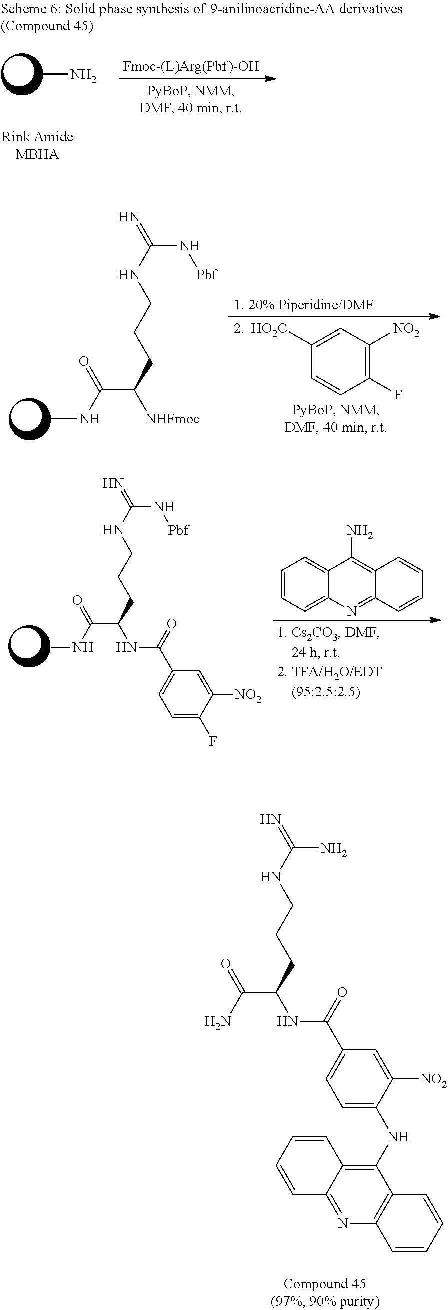

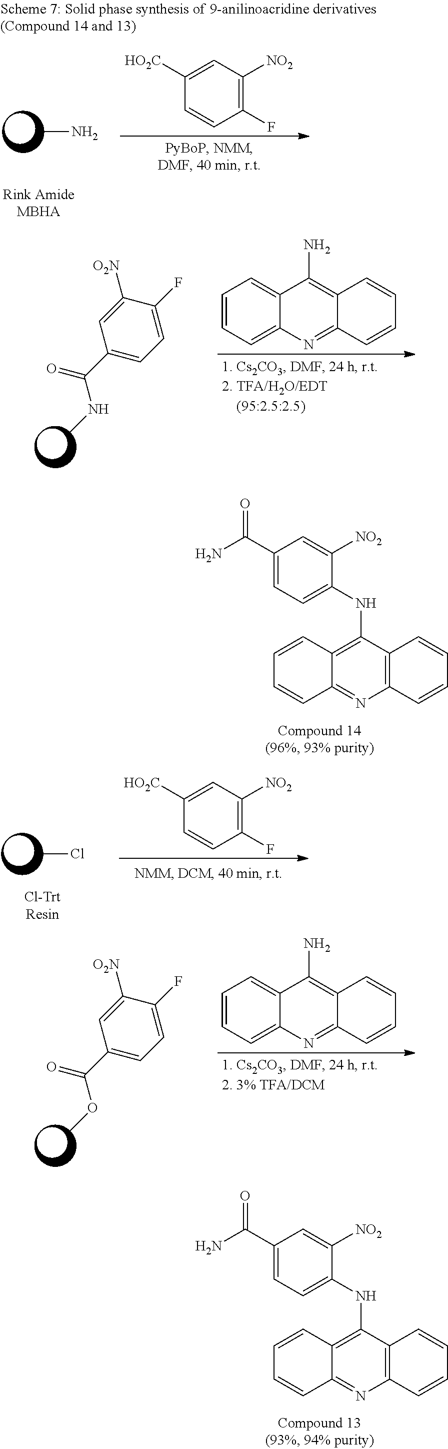

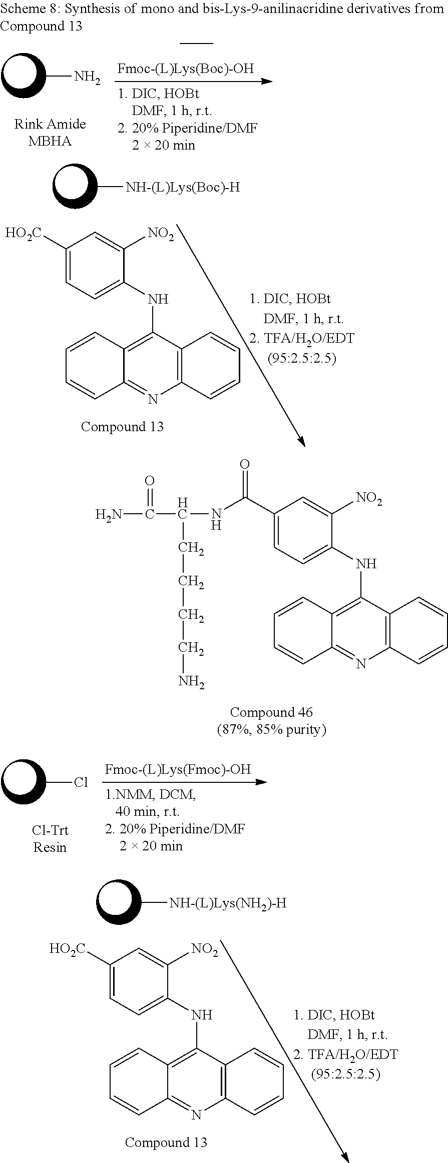

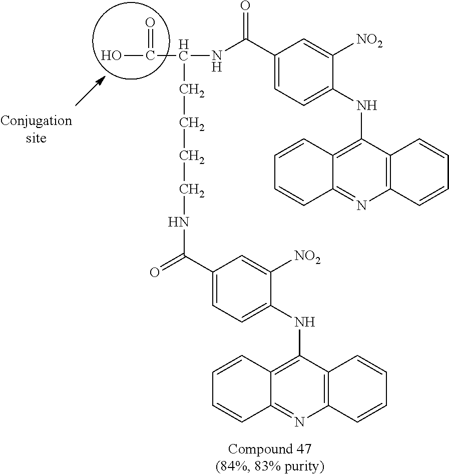

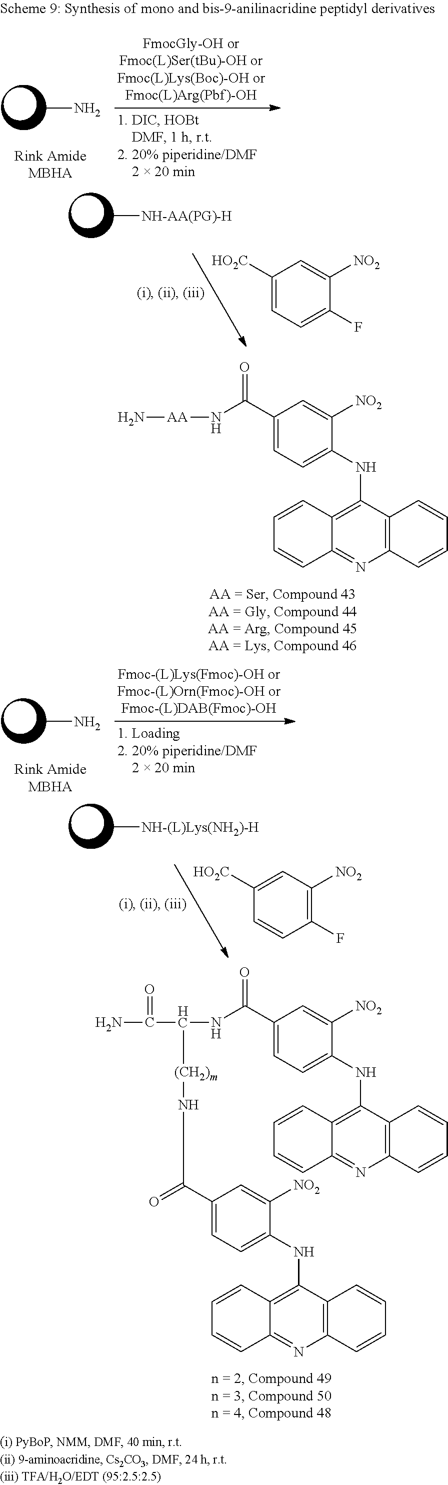

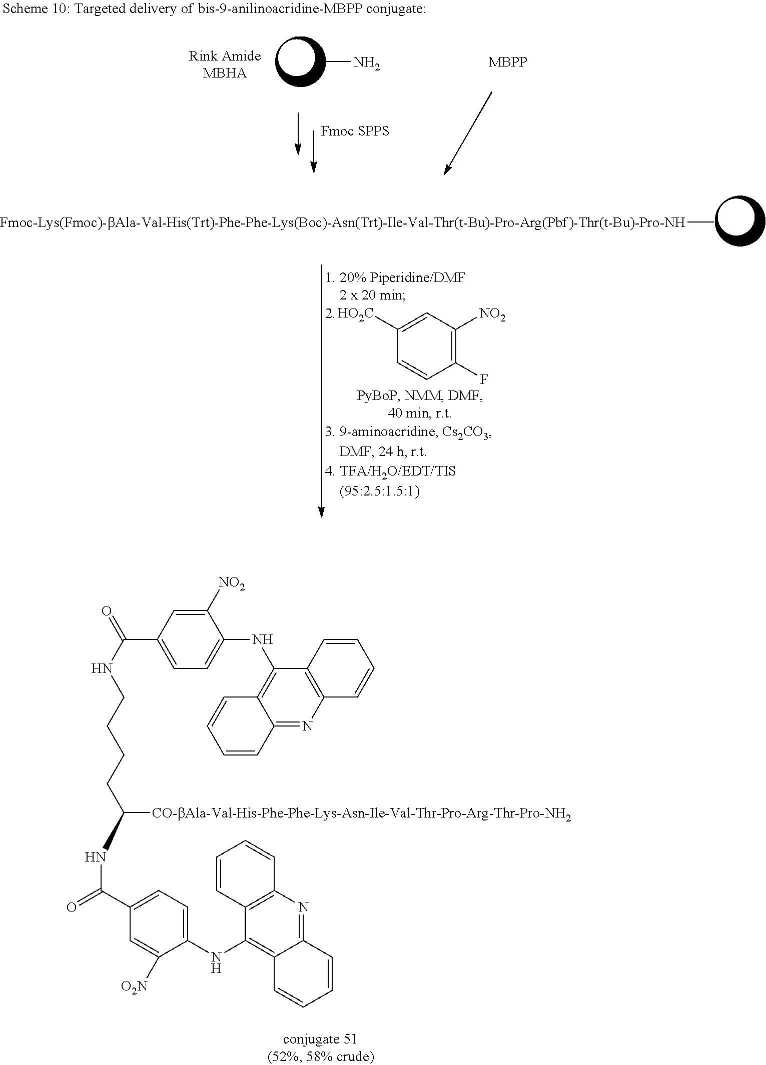

1. A 9-aminoacridine derivative of the formula I or II: 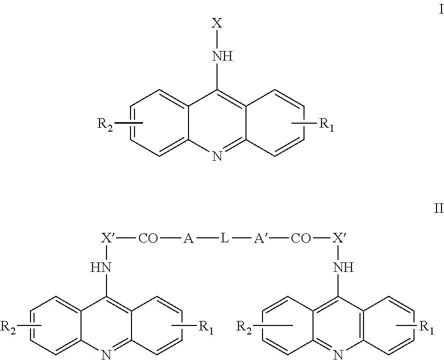
wherein
R₁ and R₂, the same or different, each is H or 1 to 2 substituents selected from electron withdrawing groups (EWG), electron donating groups (EDG), or both;
X is selected from:
(i) —CH₂-aryl or —CH₂-heteroaryl, wherein the aryl or heteroaryl is unsubstituted or substituted with one or more identical or different EWG, EDG, or both;
(ii) aryl substituted with at least one EWG and optionally further substituted with one EDG;
(iii) heteroaryl, unsubstituted or substituted with one or more EWG, one EDG, or both; or
(iv) benzoquinone or a polycyclic aromatic quinone, unsubstituted or substituted with one or more EWG, EDG, or both;
X′ is aryl or heteroaryl substituted with at least one EWG, or —CH₂-aryl or —CH₂-heteroaryl unsubstituted or substituted with one or more identical or different EWG, EDG, or both;
A and A′, the same or different, each is —NH— or —O—; and
L is a linear or branched (C₁-C₁₀)alkylene chain that may be non-adjacently interrupted by one or more N atoms or by a phenylene group, and is optionally substituted by —(CH₂)ₙ—Y, wherein n is from 0 to 10 and Y is —OH, —SH, —NH₂, —COOH, CONH₂, an amino acid residue, a (poly)peptide residue, or a polyamine residue —NH—B—NH₂, wherein B is a C₁-C₁₀ alkylene chain optionally non-adjacently interrupted by one or more N atoms;
the EWG may be selected from the groups F, Br, C₁, NO₂, CN, CF₃, SO₃H and COR₃, wherein R₃ is selected from OH; CH₂OH; (C₁-C₁₀)alkoxy; aryloxy; heteroaryloxy; a PEG moiety which may be a PEG moiety of molecular weight in the range of 200 to 40,000 Da, preferably 8,000, 10,000, or 20,000 Da; (C₁-C₁₀) alkyl; aryl; heteroaryl; a residue of an amino acid or of a derivative thereof, linked to the CO group through its α-amino group; NH₂; or a polyamine residue of the formula —NH—B—NH₂, wherein B is a C₁-C₁₀ alkylene chain optionally non-adjacently interrupted by one or more N atoms;
the EDG may be selected from (C₁-C₁₀)alkyl, OH, (C₁-C₁₀)alkoxy, or —N(R₄R₅), wherein R₄ and R₅ each independently is H or (C₁-C₁₀)alkyl;
and pharmaceutically acceptable salts thereof.

2. The 9-aminoacridine derivative of formula I or II according to claim 1, wherein the aryl is phenyl or naphthyl; heteroaryl is a mono or bicyclic group in which at least one of the rings is a 5- or 6-membered ring containing 1-3 N atoms which may be condensed to a benzo ring or to another 6-membered ring containing 1-3 N atoms such as pyrrolyl, imidazolyl, pyridyl, pyrimidyl, triazinyl, indolyl, quinolyl, isoquinolyl, benzopyrimidyl, benzopyrazyl, and pyridopyridyl, preferably pyridyl or pyrimidyl; the quinone is benzoquinone or naphthoquinone; and the amino acid is selected from the 20 naturally occurring α-amino acids, natural amino acids that are less abundant or non-natural amino acids, or a chemical derivative thereof that may be an ester or amide of the carboxy group or an N-acyl derivative of the amino group.

3. The 9-aminoacridine derivative of formula I according to claim 2 or a pharmaceutically acceptable salt thereof, wherein R₁ and R₂ are both H, and X is selected from:
(i) —CH₂-phenyl, wherein the phenyl is substituted with one EWG that may be COOH or nitro, or one EDG that may be OH, or with or one EWG that may be Br and two identical EDGs that may be methoxy, or with three identical EDGs that may be methyl or methoxy groups;
(ii) —CH₂-naphthyl substituted with one EDG that may be OH;
(iii) —CH₂-indolyl substituted with one EDG that may be methoxy;
(iv) phenyl substituted with one EWG that may be a nitro group; with two identical EWGs that may be nitro groups; with two different EWGs wherein one EWG is a nitro group and the other EWG is COOH, CONH₂, CN, or SO₃H, or one EWG is CF₃ and the other EWG is COOMe; or with one EWG that may be a nitro group and one EDG that may be OH or methoxy;
(v) pyridyl, unsubstituted or substituted with one EWG that may be a nitro or CN group;
(vi) pyrimidyl, unsubstituted or substituted with one EWG that may be Br;
(vii) benzoquinone substituted with one EWG that may be Br and one EDG that may be ethoxy; with two EWGs that may be Cl and one EDG that may be ethoxy; or with three EWGs that may be Br;
(vii) naphthoquinone substituted with one EWG that may be Cl; with one EWG that may be Cl and two EDGs that may be methyl; or with three EDGs, wherein one of them may be ethoxy and the other two EDGs are identical and may be OH; or
(viii) phenyl substituted with two different EWGs, wherein one EWG may be nitro and the other EWG may be a group COR₃, wherein R₃ is a residue of an amino acid or of a derivative thereof.

4. The 9-aminoacridine derivative of formula I according to claim 1 or a pharmaceutically acceptable salt thereof, selected from the derivatives:
(i) 2-(acridin-9-ylaminomethyl)benzoic acid, herein identified as Compound 1;
(ii) 4-(acridin-9-ylaminomethyl)benzoic acid, herein identified as Compound 2;
(iii) 2-(acridin-9-ylamino)methyl)-4-nitrophenol, herein identified as Compound 3;
(iv) N-(2-nitro-5-hydroxy-benzyl)acridin-9-amine, herein identified as Compound 4;
(v) N-(2,4,6-trimethyl-benzyl)acridin-9-amine, herein identified as Compound 5;
(vi) N-(3,4,5-trimethoxybenzyl)acridin-9-amine, herein identified as Compound 6;
(vii) N-(2-bromo-4,5-dimethoxy-benzyl)acridin-9-amine, herein identified as Compound 7;
(viii) N-(2-hydroxy-naphthylmethyl)acridin-9-amine, herein identified as Compound 8;
(ix) N-(5-methoxy-indol-3-ylmethyl)acridin-9-amine, herein identified as Compound 9;
(x) N-(2-nitrophenyl)acridin-9-amine, herein identified as Compound 10;
(xi) N-(3-nitrophenyl)acridin-9-amine, herein identified as Compound 11;
(xii) N-(2,4-dinitrophenyl)acridin-9-amine, herein identified as Compound 12;
(xiii) 4-(acridin-9-ylamino)-3-nitrobenzoic acid, herein identified as Compound 13;
(xiv) 4-(acridin-9-ylamino)-3-nitrobenzamide, herein identified as Compound 14;
(xv) 4-(acridin-9-ylamino)-3-trifluoromethyl-benzoic acid methyl ester, herein identified as Compound 15;
(xvi) 4-(acridin-9-ylamino)-3-nitro-benzonitrile, herein identified as Compound 16;
(xvii) 4-(acridin-9-ylamino)-3-nitro-benzenesulfonic acid, herein identified as Compound 17;
(xviii) N-(2,4-dinitro-5-fluorophenyl)acridin-9-amine, herein identified as Compound 18;
(xix) 5-(acridin-9-ylamino)-2-nitrophenol, herein identified as Compound 19;
(xx) N-(2-methoxy-4-nitrophenyl)acridin-9-amine, herein identified as Compound 20;
(xxi) N-(3-nitropyrid-2-yl)acridin-9-amine, herein identified as Compound 21;
(xxii) N-(pyrid-2-yl)acridin-9-amine, herein identified as Compound 22;
(xxiii) 6-(acridin-9-ylamino)nicotinonitrile, herein identified as Compound 23;
(xxiv) N-(5-methyl-3-nitropyridin-2-yl)acridin-9-amine, herein identified as Compound 24;
(xxv) N-(5-chloro-3-nitropyridin-2-yl)acridin-9-amine, herein identified as Compound 25;
(xxvi) 6-(acridin-9-ylamino)-5-nitronicotinonitrile, herein identified as Compound 26;
(xxvii) 6-(acridin-9-ylamino)-5-nitronicotinic acid, herein identified as Compound 27;
(xxviii) N-(pyrimid-2-yl)acridin-9-amine, herein identified as Compound 28;
(xxix) N-(5-bromopyrimid-2-yl)acridin-9-amine, herein identified as Compound 29;
(xxx) N-(5-methylpyrimidin-2-yl)acridin-9-amine, herein identified as Compound 30;
(xxxi) N-(5-chloropyrimidin-2-yl)acridin-9-amine, herein identified as Compound 31;
(xxxii) 2-(acridin-9-ylamino)pyrimidine-5-carbonitrile, herein identified as Compound 32;
(xxxiii) 2-(acridin-9-ylamino)pyrimidine-5-carboxylic acid, herein identified as Compound 33;
(xxxiv) 2-(acridin-9-ylamino)-3,6-dichloro-5-ethoxycyclohexa-2,5-diene-1,4-dione, herein identified as Compound 34;
(xxxv) 2-(acridin-9-ylamino)-3-bromo-5-ethoxy-benzoquinone, herein identified as Compound 35;
(xxxvi) 2-(acridin-9-ylamino)-3,6-dibromo-5-ethoxycyclohexa-2,5-diene-1,4-dione, herein identified as Compound 36;
(xxxvii) 2-(acridin-9-ylamino)-3-chloro-5-ethoxycyclohexa-2,5-diene-1,4-dione, herein identified as Compound 37;
(xxxviii) 2-(acridin-9-ylamino)-3,5,6-tribromobenzoquinone, herein identified as Compound 38;
(xxxix) 2-(acridin-9-ylamino)-3-chloronaphthoquinone, herein identified as Compound 39;
(xxxx) 2-(acridin-9-ylamino)-3-chloro-6,7-dimethylnaphthoquinone, herein identified as Compound 40; and
(xxxxi) 2-(acridin-9-ylamino)-3-ethoxy-5,8-dihydroxy-naphthoquinone, herein identified as Compound 41.

5. The 9-aminoacridine derivative of formula I according to claim 3, wherein X is as defined in paragraph (viii) and wherein the amino acid is glycine or a trifunctional amino acid selected from serine, lysine and arginine, and the amino acid may be in the form of a derivative, preferably the amide (CONH₂).

6. The 9-aminoacridine derivative of formula I according to claim 5, wherein:
(i) the amino acid is serine and the derivative is 2-(4-(acridin-9-ylamino)-3-nitrobenzamido)-3-hydroxypropanoic acid, herein identified as Compound 42.
(ii) the amino acid derivative is serinamide and the compound is 4-(acridin-9-ylamino)-N-(1-amino-3-hydroxy-1-oxoprop-2-yl)-3-nitrobenzamide, herein identified as Compound 43;
(iii) the amino acid derivative is glycinamide and the compound is 4-(acridin-9-ylamino)-N-(2-amino-2-oxoethyl)-3-nitrobenzamide, herein identified as Compound 44;
(iv) the amino acid derivative is argininamide and the compound is 4-(acridin-9-ylamino)-N-(1-amino-5-guanidino-1-oxopent-2-yl)-3-nitro-benzamide, herein identified as Compound 45; and
(v) the amino acid derivative is lysinamide and the compound is 4-(acridin-9-ylamino)-N-(1,6-diamino-1-oxohex-2-yl)-3-nitrobenzamide, herein identified as Compound 46.

7. The 9-aminoacridine derivative of formula II according to claim 1, wherein R₁ and R₂ are both H, each X′ is phenyl substituted with one EWG that may be nitro, A and A′ are both —NH— and L is a linear C₄-C₅-alkylene chain substituted at the C atom adjacent to A or A′ with a group COOH or CONH₂.

8. The 9-aminoacridine derivative of formula II according to claim 7, wherein:
(i) the derivative is 2,6-bis(4-(acridin-9-ylamino)-3-nitrobenzamido)hexanoic acid, herein identified as Compound 47;
(ii) the derivative is N,N′-(6-amino-6-oxohexane-1,5-diyl)bis-(4-(acridin-9-ylamino)-3-nitro-benzamide), herein identified as Compound 48;
(iii) the derivative is N,N′-(4-amino-4-oxobutane-13-diyl)bis(4-(acridin-9-ylamino)-3-nitrobenzamide), herein identified as Compound 49; and
(iv) the derivative is N,N′-(5-amino-5-oxopentane-1,4-diyl)bis(4-(acridin-9-ylamino)-3-nitrobenzamide), herein identified as Compound 50.

9. The 9-aminoacridine derivative of formula II according to claim 7, wherein R₁ and R₂ are both H, each X′ is phenyl substituted with one EWG that may be nitro, A and A′ are both —NH—, and L is a linear C₅-alkylene chain substituted with a peptide residue having 10-20 amino acid residues.

10. The 9-aminoacridine derivative of formula II according to claim 9, wherein said peptide residue has the sequence herein identified as SEQ ID NO:2 and the derivative is herein identified as Compound 51.

11. A pharmaceutical composition comprising a 9-aminoacridine derivative or a pharmaceutically acceptable salt thereof according to claim 1 and a pharmaceutically acceptable carrier.

12. The pharmaceutical composition according to claim 11, wherein said 9-aminoacridine derivative is the compound herein identified as Compound 41 or a pharmaceutically acceptable salt thereof.
